(19) United States
(12) Patent Application Publication
(10) Pub. No.: US 2016/0303236 A1
(43) Pub. Date: Oct. 20, 2016

(54) REGIONALLY ACTIVATED DRUG DELIVERY NANOPARTICLES

(71) Applicant: THE REGENTS OF THE UNIVERSITY OF CALIFORNIA, Oakland, CA (US)

(72) Inventors: Sadik C. Esener, Solana Beach, CA (US); Carolyn Schutt Ibsen, San Diego, CA (US); Stuart Ibsen, San Diego, CA (US); Eran Zahavy, Rehovot (IL)

(21) Appl. No.: 15/102,227

(22) PCT Filed: Dec. 10, 2014
(86) PCT No.: PCT/US14/69496

§ 371 Date: Jun. 6, 2016

Related U.S. Application Data
(60) Provisional application No. 61/914,376, filed on Dec. 10, 2013.

(51) Int. Cl. A61K 41/00; A61K 31/704; A61K 31/519; A61K 47/48

(52) U.S. Cl. CPC A61K 41/0042; A61K 47/48061; A61K 31/704; A61K 47/48884; A61K 31/519

(57) ABSTRACT

Methods, systems, and devices are disclosed for systemic delivery and selective and localized activation of nanoparticles containing an agent in an inactive form or a prodrug in a subject.

### CROSS-REFERENCE TO RELATED APPLICATIONS

This application claims priority to and benefits of U.S. provisional application No. 61/914,376 entitled “REGIONALLY ACTIVATED DRUG DELIVERY NANOPARTICLES” filed on Dec. 10, 2013, the content of which is incorporated by reference in its entirety.

### STATEMENT REGARDING FEDERALLY SPONSORED RESEARCH OR DEVELOPMENT

This invention was made with government support under Grant Numbers, T32 CA121938, R25 CA153915, and U54 CA119335, awarded by the National Cancer Institute (NCI) of the National Institutes of Health (NIH). The government has certain rights in the invention.

### TECHNICAL FIELD

This patent document relates to nanoscale materials and nanotechnologies in drug delivery.

### BACKGROUND

Nanotechnology provides techniques or processes for fabricating structures, devices, and systems with features at a molecular or atomic scale, e.g., structures in a range of one to hundreds of nanometers in some applications. For example, nano-scale devices can be configured to sizes within one hundred to ten thousand times smaller than human cells, e.g., similar in size to some large biological molecules (biomolecules) such as enzymes and receptors. Nano-sized materials used to create a nanostructure, nanodevice, or a nanosystem that can exhibit various unique properties that are not present in the same materials scaled at larger dimensions and such unique properties can be exploited for a wide range of applications.

### SUMMARY

Techniques, systems, and devices are disclosed for a drug delivery platform based on a nanoparticle having light-activatable prodrug. The drug delivery platform can precisely deliver the prodrug to a desired tissue region of a subject, such as a tumor, in its prodrug form. The nanoparticles can be administered systemically via intravenous injection, infusion, or ingestion, an activating light can then be delivered specifically to the desired region, such that the prodrug will only be converted to the toxic active therapeutic form when exposed to the delivered light.

In one aspect, a chemical delivery system includes a nanoparticle structured to include a chemical substance that is chemically inactive and capable of being transformed or converted to a chemically active substance caused by an applied optical stimulus, in which the particle is configured to be injected, infused, or ingested by a subject and move through the subject's bloodstream to regions of the subject's body, and a light emitting device to emit light having a particular wavelength at a selected tissue region of the subject, in which exposure to the emitted light on the particle at the selected tissue region chemically activates the chemical substance to interact with the selected tissue region.

The subject matter described in this patent document and attached appendices can be implemented in specific ways that provide one or more of the following features. For example, the disclosed technology can be implemented to create self-assembled nanoparticles that include prodrug monomers, or having cores that include prodrug monomers, which are activated by light delivered to a specific tissue region using, for example, fiber optic and light emitting diode technology. Applications of the disclosed technology can include, but are not limited to, chemotherapy treatment for cancer where the general location of the tumor and metastatic sites are known; medical conditions where the region of desired activation is restricted to localized tissue regions and it is desirable to limit exposure of the active therapeutic to the tissue regions outside these areas; and therapeutics that are meant to treat infections (e.g., bacterial, viral, fungal) that are localized to regions of the body and it is desired to prevent systemic exposure to the therapeutic agents; among others.

The disclosed technology in this patent document relates to systemic administration of nanoparticles of an inactive agent or a prodrug to a subject, and subsequently upon localized photoactivation, the inactive agent or the prodrug is converted into an active therapeutic agent or drug in the desired tissue, organ, or body part.

In one aspect, a delivery device is provided to include a nanoparticle comprising monomers of an agent in an inactive form, wherein upon exposure to an optical stimulus, the agent is activated. In one embodiment, the nanoparticle has a diameter of less than about 200 nm. In implementations, the nanoparticles have an extended circulation life in the subject upon administration in comparison to the free monomers of the agent. In implementations, the monomers may be structured to include balanced hydrophobic and hydrophilic regions to allow the monomers to self-assemble into nanoparticles. In certain implementations, the nanoparticles can include a surface coating.

In some embodiments, the nanoparticle comprises an inactive agent or a prodrug that is photo-activatable upon exposure to an optical stimulus. For example, the active component or the active drug of the inactive agent or the prodrug is covalently linked to a substituent that inhibits the activity of the active component or the active drug via a photocleavable linker. In some embodiments, the inactive agent or the prodrug is activated upon exposure to an optical stimulus by cleaving the linker and releasing the active component or drug. In implementations, the optical stimulus is specifically delivered to a desired tissue, organ, or body part. For example, the optical stimulus is delivered via LED or fiber optic technology.

In one embodiment, the active drug is doxorubicin, converted from the prodrug upon exposure to light having a wavelength of 365 nm.

In a related aspect, the disclosed technology relates to a process that allows self-assembling of monomers of an inactive agent or a prodrug into nanoparticles. In one embodiment, the process is a nanoprecipitation process.

The disclosed technology also includes a composition comprising nanoparticles, wherein each nanoparticle comprises monomers of an agent in an inactive form, which is capable of being activated upon exposure to an optical stimulus. In some embodiments, the composition further comprises a pharmaceutically acceptable carrier, excipient, surfactant, preservative, or a combination thereof.

In one embodiment, the nanoparticle has a diameter of less than about 200 nm. In implementations, the nanoparticles can be configured to have an extended circulation life in the subject upon administration in comparison to the free monomers of the agent. In implementations, the monomers can be structured to include balanced hydrophobic and hydrophilic regions to allow the monomers to self-assemble into nanoparticles. In some embodiments, the nanoparticles can include a surface coating.

In some embodiments, the nanoparticle comprises an inactive agent or a prodrug that is photo-activatable upon exposure to an optical stimulus. For example, the active component or the active drug of the inactive agent or the prodrug is covalently linked to a substituent that inhibits the activity of the active component or the active drug via a photocleavable linker. In some embodiments, the inactive agent or the prodrug is activated upon exposure to an optical stimulus by cleaving the linker and releasing the active component or drug. In implementations, the optical stimulus can be specifically delivered to a desired tissue, organ, or body part. For example, the optical stimulus is delivered via LED or fiber optic technology.

In one embodiment, the active drug is doxorubicin, converted from the prodrug upon exposure to light having a wavelength of 365 nm.

In another aspect, a method is provided for delivering an active agent to a desired tissue, organ, or body part of a subject, comprising: (a) systemically administering to the subject a composition comprising nanoparticles, wherein each nanoparticle comprises monomers of an agent in an inactive form, and subsequently, (b) exposing the desired tissue, organ, or body part of the subject to an optical stimulus, whereby the agent is activated upon exposure to the optical stimulus.

In some embodiments, the composition is administered to the subject by injection, infusion or ingestion. Any means of administration, including intravascular injection, that allows the nanoparticles to freely move in the blood circulation to reach a desired tissue or region in the subject's body can be used. In addition, exemplary administration routes such as intraperitoneal injection and subcutaneous injection can be used to allow the nanoparticles to reach additional regions in the body.

In one embodiment, the nanoparticle has a diameter of less than about 200 nm. In implementations, the nanoparticles can be configured to have an extended circulation life in the subject upon administration in comparison to the free monomers of the agent. In implementations, the monomers can be structured to include balanced hydrophobic and hydrophilic regions to allow the monomers to self-assemble into nanoparticles. In some embodiments, the nanoparticles comprise a surface coating.

In some embodiments, the nanoparticle comprises an inactive agent or a prodrug that is photo-activatable upon exposure to an optical stimulus. For example, the active component or the active drug of the inactive agent or the prodrug is covalently linked to a substituent that inhibits the activity of the active component or the active drug via a photocleavable linker. In some embodiments, the inactive agent or the prodrug is activated upon exposure to an optical stimulus by cleaving the linker and releasing the active component or drug. In implementations, the optical stimulus can be specifically delivered to a desired tissue, organ, or body part. For example, the optical stimulus is delivered via LED or fiber optic technology.

In one embodiment, the active drug is doxorubicin, converted from the prodrug upon exposure to light having a wavelength of 365 nm.

### BRIEF DESCRIPTION OF THE DRAWINGS

FIG. 1 shows a diagram depicting the molecular structure of DOX-PCB. The exemplary DOX molecule is attached to a photocleavable nitrophenol compound that has a short polyethylene glycol linker attached to a biotin molecule at the opposite end.

FIG. 2 shows a three dimensional molecular diagram of the conformation of the exemplary DOX-PCB prodrug monomer.

FIG. 3 shows a schematic of the exemplary nanoparticle of DOX-PCB monomers. The hydrophobic regions of the monomer are denoted in black and the hydrophilic regions are denoted in gray. The hairpin structure of the monomer pairs the hydrophobic regions together allowing the hydrophilic tail to extend beyond the bend. When nanoprecipitated, the monomers cluster to form a core with the biotin tails covering the surface.

FIG. 4 shows a schematic representation of the exemplary concept of nanoparticle delivery of light-activatable prodrug followed by light activation in a localized tissue region (e.g., a tumor).

FIG. 5 shows a scanning electron microscope (SEM) image of exemplary DOX-PCB nanoparticles produced by the exemplary nanoprecipitation process of the DOX-PCB monomer.

FIG. 6 shows that the concentration of DOX converted from the DOX-PCB nanoparticles increased linearly with the duration of 365 nm light exposure (R²=0.93).

FIG. 7 shows light microscopy images of DOX and DOX-PCB nanoparticle localization within live PTK2 kidney epithelial cells. Panels A, C, and E are phase contrast images of the cells and panels B, D, and F are fluorescent images of the same field of view as the panel directly above. Panels C, D, E, and F show the same group of cells before and after 365 nm light exposure. (A, B) DOX is seen to associate strongly with the cell nuclei. (C, D) The cells receiving DOX-PCB nanoparticles showed fluorescenceinside the cells but not associated with the nuclei. (E, F) After 365 nm light exposure, the nuclei from the same cells shown in C and D show increased fluorescence supporting that free DOX was released from DOX-PCB.

FIG. 8 shows cytotoxicity results on human lung cancer cells of DOX-PCB prodrug nanoparticles, photoactivated DOX-PCB nanoparticles and free DOX. DOX-PCB prodrug nanoparticles show significantly reduced toxicity from free DOX, with DOX-PCB yielding a 30-fold higher IC50 value. The DOX-PCB nanoparticles that were photo-activated for 60 minutes displayed an increased cellular toxicity compared to unexposed DOX-PCB nanoparticles.

FIG. 9 shows a transmission electron micrograph (TEM) image of exemplary self-assembled nanoparticles prepared with a core of DOX-PCB prodrug and a surface coat of lipid and lipid-polymer conjugate.

FIG. 10 shows a schematic diagram of the microscope system and sample holder setup used to observe and record the interaction of the fluorescent microbubbles with the focused ultrasound.

FIG. 11 shows still frames from videos showing examples of the three categories of debris cloud expansion. A. Two fluorescent microbubbles are shown before ultrasound exposure with an edge-to-edge distance of 36 μm. B. The two microbubbles did not appear to interact with each other during cavitation and the resulting debris clouds from cavitation are both radial in shape as shown in frame C. D. Here the microbubbles have an edge-to-edge distance of 8.0 μm before ultrasound exposure. E. The microbubbles interacted with one another and distorted the shape of the resulting two debris clouds created by the cavitation shockwave to be ellipsoid in shape as shown in frame F. G. Here the two microbubbles have edge-to-edge distance of 9.3 μm before ultrasound exposure. H. The microbubbles interacted with each other during cavitation and a horizontal directional component of the jet was formed resulting in a long distortion of both of the lipid debris clouds. I. The dimensions depicted indicate two elongated debris clouds with lengths greater than 2 times their heights.

FIG. 12 shows that three microbubbles are in physical contact with one another before ultrasound exposure. Upon exposure to ultrasound the resulting elongated lipid debris cloud shows the formation of a horizontal directional component in the fluid jet. All other microbubble pairs or triplets in physical contact prior to ultrasound exposure were also observed to form elongated debris clouds.

FIG. 13 shows box plots of the distance between the microbubble and its nearest neighbor in microns for each of the three debris cloud expansion categories. Whiskers denote maxima and minima and the median is represented as the horizontal line. Outliers were defined as any points that were two standard deviations away from the mean and are indicated as open circles. The radial and elongated debris cloud categories and the ellipsoid and elongated categories were found to be statistically significant from each other (p<0.001 and p=0.041, respectively) with regard to inter-microbubble distance. The difference in inter-bubble distances resulting in radial and ellipsoid debris clouds trended toward significance (p=0.067).

FIG. 14 shows box plots for the diameter of the microbubbles in each of the three debris cloud expansion categories. Whiskers denote maxima and minima and the median is represented as a horizontal line. Outliers were defined as any points that were two standard deviations away from the mean and are indicated as open circles.

FIG. 15 scatter plot showing the diameters of the microbubbles plotted against distance between the microbubble and its nearest neighbor. Points are color-coded to the shape of the debris cloud. A trend is observed where radial expansion is seen at larger inter-microbubble distances and ellipsoid and elongated debris cloud formation are seen at the shorter distances.

### DETAILED DESCRIPTION

### I. Introduction

Implementations or examples of embodiments of the technology disclosed in this document provide various details of disclosed nanoscale materials and nanotechnologies in drug delivery that are specific to the described implementations or examples.

One described example is a chemical delivery system that includes a nanoparticle comprising a chemical substance that is chemically inactive and capable of converting to a chemically active substance caused by an applied optical stimulus. This nanoparticle is configured to be capable of being injected, infused, or ingested by a subject and is able to move through the subject's bloodstream to regions of the subject's body. This system includes a light emitting device to emit light having a particular wavelength at a selected tissue region of the subject. The particular wavelength is selected so that the exposure to the emitted light on the nanoparticle at the selected tissue region chemically activates the chemical substance to interact with the selected tissue region. The light is delivered to the selected tissue, region, or body part in a controlled manner such that other tissues, regions, or body parts are not exposed to the light.

In describing such implementations or examples, an “individual,” “subject,” “patient,” or “user” includes a vertebrate, for example, a mammal, including a human. Examples of mammals include, but are not limited to, humans, domestic and farm animals, and zoo, sport or pet animals. A “chemical substance” includes a chemical compound that can effectuate a therapeutic effect to a condition. In some implementations, such a chemical substance may have inactive and active forms and a particular therapeutic effect may also be available when the substance is in its active form. An “agent” includes a substance or one or more molecules that can effectuate a therapeutic effect to a condition. In some implementations, such an agent may have inactive and active forms and a particular therapeutic effect may be available when the agent is in its active form. Examples of such agents include, but are not limited to, chemical compounds, small molecules, nucleic acids, peptides, lipids, antibodies, signaling molecules, etc. A “pharmaceutically acceptable carrier, excipient, surfactant, or preservative” includes non-toxic, inert solvents, dispersion media, coatings, antibacterial and antifungal agents, isotonic and absorption delaying agents, excipients, diluents, surfactants, preservatives, and the like, compatible with pharmaceutical administration. Suitable carriers, surfactants and preservatives are described in the most recent edition of Remington's Pharmaceutical Sciences.

### II. Nanoparticles as Drug Delivery Vehicles

Cancer is a disease of uncontrolled cellular replication that occurs simultaneously with healthy cell replication. Most chemotherapy drugs take advantage of cancer's uncontrolled growth by interfering with some aspects of replication. A main goal for chemotherapy development is to allow systemically distributed drugs to differentiate healthy replication from tumor replication to prevent the dose-limiting side effects experienced by patients.

To address this problem, the field of drug delivery looks to physically encapsulate these drugs in a vehicle which carries its payload systemically through healthy tissue with minimal release. However, these vehicles need to release their payload in the tumor tissue either by preferential accumulation with slow drug release or by a tumor-specific triggering event.

Nanoparticles are used as vehicles for delivering chemotherapy drugs. The small particle size range of the nanoparticles allows longer circulation time such that the chemotherapy drug can passively extravasate through discontinuous endothelium in the tumor. The systemically circulating nanoparticles pass through healthy tissues having continuous endothelium with minimal accumulation, while preferentially accumulating within the “leaky” vasculature of the tumor having discontinuous endothelium.

A major challenge facing nanoparticle-based drug delivery vehicles with chemotherapy payloads is accumulation in healthy tissues through passive extravasation as well as active uptake by the reticulo-endothelial systems. In addition to the tumor, nanoparticles also accumulate in certain healthy tissues of the body, such as the creases of the skin on the hands and feet where it is thought that constant motion causes natural microdamage to the blood vessels allowing nanoparticles to extravasate. Additionally, nanoparticles may accumulate in the bone marrow through the reticulo-endothelial system (RES), as well as by extravasation due to the bone marrow's discontinuous endothelium.

An example is FDA-approved Doxil®, the liposomal formulation of the chemotherapy drug doxorubicin (DOX). Doxil® particles dramatically change the pharmacokinetics and biodistribution of DOX due to their particle size range of between 80 nm and 120 nm and polyethylene glycol (PEG) surface coatings. Free DOX has a circulation half-life of 15-20 minutes while Doxil® has a half-life of 42 hours. Doxil® accumulated in the tumor slowly releases the payload through dissolution of the internal crystallized DOX. However, the skin ulceration or irritation resulting from the DOX release in the hands and feet is known as Palmar-plantar erythrodysesthesia and is a major dose-limiting side effect. Moreover, the bone marrow cells have a very high replication rate and are very sensitive to DOX, which affects replication. The released payload from Doxil® accumulated in the bone marrow causes dose-limiting myelosuppression.

The unintentional accumulation of Doxil® in healthy tissues is a major dose-limiting concern. There is no particle size window that avoids extravasation in healthy tissues while allowing long circulation time. Although PEG surface coatings can delay accumulation, especially in the RES, the Doxil® particles still accumulate in the healthy tissues over time.

### III. Prodrugs with Controllable Photolysis Activation

Prodrugs are covalently modified versions of the active drug that display significantly reduced toxicity from the original active drug but have the ability to be restored to a therapeutic form when triggered. If the trigger is tumor-specific then the prodrug is converted to its active form in the tumor with substantially lower toxic side effects.

A prodrug using a photocleavable linker covalently bound to DOX to render it less toxic to tissue has recently been developed. See Ibsen et al., Pharm. Res. 27: 1848-1860 (2010). The prodrug of DOX has the unique ability to restore full therapeutic function when photo-triggered. Active DOX was shown to be released inside tumors in vivo when the activating light was delivered to the center of the tumor using a fiber optic. See Ibsen et al., Photochemistry and Photobiology 89(3): 698-708 (2013).

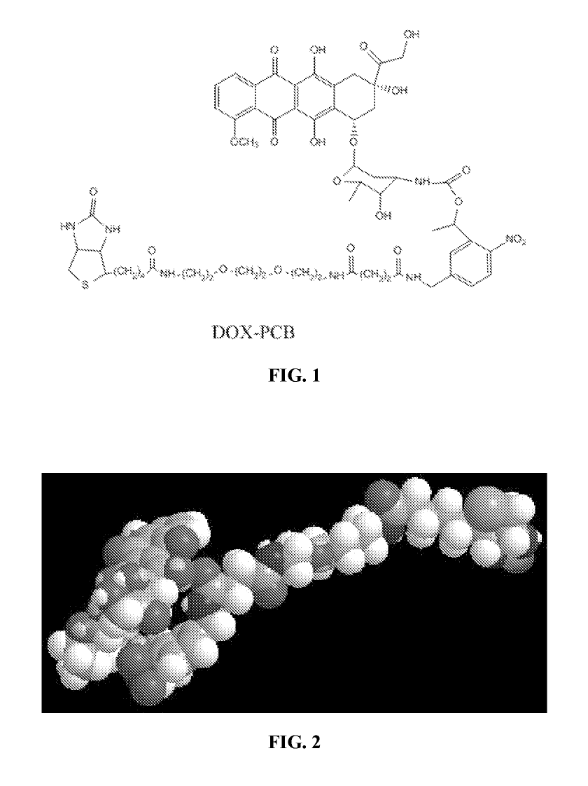

The prodrug compound, called DOX-PCB, was created by blocking the free amine located on the sugar moiety with a nitrophenyl group conjugated to a short polyethylene glycol linker and terminated with a biotin (PCB) as shown in FIG. 1.

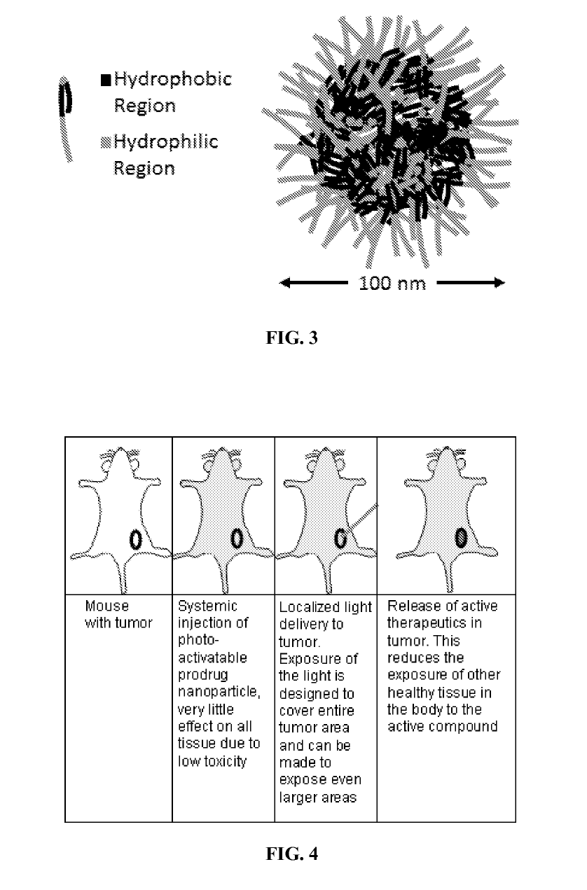

Three dimensional modeling of the molecular conformation of DOX-PCB in water is shown in FIG. 2. This hairpin shape results from the hydrophobic aglycone structure of DOX interacting with the hydrophobic nitrophenyl group. This gives the molecule a hydrophobic region and a hydrophilic tail region consisting of the PEG linker and biotin.

The choice of the prodrug trigger that restores the drug to its therapeutic form is critical to assure tumor specificity. The use of light is different from traditional activating stimuli that rely on the biochemistry of the tumor. These biochemical triggers include differences in the microenvironment between tumor and normal tissue such as tumor-associated hypoxia or low pH, as well as enzymatic cleavage by enzymes which the tumor over-expresses. The drawback is that these biochemical triggers are often present in both tumor and non-tumor tissue, especially in the liver where there is a high level of enzymatic activity.

The light-based trigger can achieve a higher level of specificity than biochemical triggers because the activating wavelength of light can be delivered specifically to the tumor tissue without exposing other tissue, region, or body part to the light using light emitting diode (LED) technology. Recent developments in LED manufacturing enable elements to be made as thin as a human hair, allowing them to be inserted or implanted anywhere a biopsy needle, endoscope, or catheter can go. The photocleavable linker in DOX-PCB is resistant to metabolic degradation which prevents undesired activation of the prodrug in healthy tissue regions, including the liver, but is activated by exposure to 365 nm light. This wavelength showed low absorption by internal tissue including DNA but had high absorption by melanin, thereby preventing external sources from causing uncontrolled release within the body.

Although DOX-PCB was resistant to metabolic activation in vivo, DOX-PCB had an a circulation half-life of 10 minutes, comparable to that of DOX at about 20 minutes. See Ibsen et al., Photochemistry and Photobiology 89: 698-708 (2013).

### IV. Nanoparticles of Photoactivatable Prodrugs

Techniques, systems, and devices are described herein for a drug delivery platform that is based on a nanoparticle comprising a light-activatable prodrug. The disclosed drug delivery platform is implemented using the nanoparticle formulation of the light-activatable prodrug to precisely deliver an active drug to the desired tissue region, such as a tumor. In some implementations, for example, the nanoparticles are administered systemically via injection (such as intravascular injection, intraperitoneal injection, and subcutaneous injection), infusion, or ingestion. Subsequently, the activating light is delivered specifically to the desired tissue region (e.g., tumor) using existing light emitting diode (LED) and/or fiber optic technology. The disclosed delivery platform is implemented such that the prodrug is only converted to a fully active therapeutic form (e.g., toxic to the desired tissue) when exposed to the delivered light. For example, the light exposure can keep the activation region spatially localized, reducing wide systemic exposure.

A. Nanoparticles of Prodrug Monomers

The design of the prodrug monomer is important for the disclosed drug delivery platform. The monomer as a whole is configured to have a correct balance of hydrophobic and hydrophilic components in the correct orientations within the monomer to allow it to self-assemble into the nanoparticle or nanoparticle core. This configuration avoids the need for any additional scaffold or filler material in the nanoparticle, thereby to allow the maximum amount of prodrug to be carried within the nanoparticle. This design also allows for high prodrug entrapment percentages, e.g., thereby decreasing the waste of prodrug in the manufacturing process. A high drug entrapment percentage can be achieved due to the covalent bond between the drug and the photocleavable component causing both to act as a single prodrug monomer unit during the nanoprecipitation procedure. Pure drug are not released from the nanoparticle in significant amounts unless exposed to the activating wavelength of light. The light activatable portion of the prodrug monomer is also critical because it allows the drug portion to be released from the prodrug monomer, not by biochemical methods, but by controlled exposure to the activating wavelength of light only. This whole scheme allows the drug to be efficiently loaded into a nanoparticle and its release controlled by the exposure to light of a triggering wavelength to a specific tissue, organ, or body part without exposing other neighboring tissue, organ, or body part.

There are many different therapeutic agents or drugs, photocleavable linkers, and substituents that can be combined to have the above required properties to form nanoparticles in various implementations. For example, suitable drugs are hydrophilic, hydrophobic, or a combination thereof and can be paired with appropriate linkers and substituents that give the overall monomers the ability to self-assemble into nanoparticles, e.g., by nanoprecipitation or by other manufacturing means.

### Therapeutic Agents

In some examples, many different drugs can be used to form the drug portion of the prodrug monomer which forms the nanoparticles. This can be beneficial because well characterized and well understood drugs can be made into prodrugs. For example, the applications can include any medical condition where it is desirable that the fully active therapeutic compound be limited in its systemic exposure except in the desired tissue region. Examples can include, but are not limited to:

(a) chemotherapy treatment for cancer, including inoperable tumors;
(b) localized chemotherapy in the region of a resected tumor to treat any tumor margins left behind;
(c) localized chemotherapy treatment of a tumor before resection to help reduce the possibility of tumor margins being left behind or the inadvertent release of metastatic cells from the tumor during the surgery;
(d) antibiotic treatment for localized bacterial infections, including drug resistant strains of bacteria;
(e) localized treatment of viral infections, including drug resistant strains of virus;
(f) localized treatment for fungal infections, including drug resistant strains of fungus;
(g) localized activation of a drug in certain brain regions to prevent brain-wide exposure;
(h) localized activation of monoclonal antibody therapies such as, but not limited to, ipilimumab, which is a monoclonal antibody cancer therapy;
(i) localized activation of immunologic adjuvants which can be used to stimulate the immune system for the treatment of cancer or other medical conditions;
(j) eye applications where the drug needs to be active in the eyes but not the rest of the body, including drugs that affect vascular growth;
(k) wound healing applications where compounds are needed internally or topically to encourage and enhance wound healing without affecting other tissues;
(l) localized immunosuppression for transplantation applications;
(m) localized hormone treatments;
(n) bone healing treatments;
(o) vascular clot and plaque removal;
(p) localized sealing of blood vessels to stop bleeding;
(q) enhancing localized vascular growth; and/or
(r) treatment of lymph nodes including treatment of lymph nodes containing metastatic cancer cells or prophylactic treatment of lymph nodes to prevent accumulation of metastatic cells.

In some examples, multiple therapeutic agents are released from the same prodrug monomer. There can be additional therapeutic agents in the monomer that are activated upon light exposure. There can be a primary therapeutic agent that is released from the prodrug monomer and the other chemical byproducts can be therapeutic as well. Multiple drugs could be conjugated by photocleavable linkers to the same monomer. This allows for simultaneous release or activation of multiple drugs from the nanoparticle at a localized activation site. Monomers do not necessarily have to be linear in nature, branched structures could also be used here as long as they are capable of self-assembling into nanoparticles.

### Linkers

In one example, the prodrug monomers can use many different photocleavable linker compounds that can be activated by different wavelengths of light capable of releasing the pure drug or the drug with minor substituents still attached to it. The use of infrared activated prodrugs can allow for larger regions of tissue to be in the activation zone. This can also allow the light sources to be located externally to the body and still reach deep tissue.

For example, the photocleavable linkers may be designed to work for this purpose and release active drug. In exemplary implementations, for example, a 4,5-Dimethoxy-2-nitrobenzyl chloroformate (NVOC) is used, which conjugates with DOX, but when photoactivated it causes only non-functional forms of DOX to be released.

### Substituents

In some examples, the monomers can have many different substituent compounds attached to the opposite end of the photocleavable linker from the drug including polyethylene glycol monomers of different lengths. The major property that the monomers must have is the correct balance of hydrophobic and hydrophilic portions that are located in the correct positions within the monomer to allow the monomers to form the entire nanoparticle or the core of the nanoparticle.

### Surface Coatings

In some examples, the prodrug monomers can be used to form the core of a nanoparticle which is covered with a surface coating. The surface coatings may include but are not limited to: phospholipids, polymers, lipid-polymer conjugates, or any combination of the above. Such surface coatings may be used to prolong the circulation time of the nanoparticle and/or reduce adhesion of surface proteins. It should be noted these functionalities can also be achieved without the need of a surface coating by designing them into the photocleavable monomer itself. Additional surface layers can be added to the nanoparticles to provide different functionalities, but in such exemplary cases, the core of the nanoparticle entirely or substantially comprises pure prodrug. The surface coating can be used to add targeting functionality, for example through the use of targeting peptides or antibodies which can be conjugated to the lipids or polymers that form the surface coat. The surface coating can also be used to incorporate imaging contrast agents onto the surface of the particle for visualization with MRI, ultrasound, optical, or other modes of imaging. The surface coating may also be used to incorporate moieties onto the prodrug nanoparticle that promote cellular internalization or endocytosis such as folate.

### Particle Size

The nanoparticle formulation of the prodrug monomers has a certain particle size to improve the circulation time, pharmacokinetics, and biodistribution over what is achieved by the free prodrug monomers not in a nanoparticle form. The diameter of the nanoparticles is about 200 nm or less than 200 nm, such as less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In some embodiments, the nanoparticles have a diameter of between 40 nm and 200 nm to achieve an ideal prolonged circulation time.

### Exemplary DOX-PCB Nanoparticle

The exemplary design for the formation of the disclosed nanoparticle includes using light-activatable prodrug monomers that are capable of self-assembling into nanoparticles. The following exemplary prodrug monomer, referred to as DOX-PCB, has been used in exemplary implementations of the disclosed technology. The molecular structure of DOX-PCB is shown in FIG. 1. An exemplary computer model of the three-dimensional conformation of the prodrug monomer is shown in the diagram of FIG. 2. The DOX-PCB molecule takes on a hairpin configuration in water that creates a hydrophobic character on one end of the molecule and a hydrophilic region on the other side. The monomer has the correct balance of hydrophobic and hydrophilic components in the correct location within the monomer to form nanoparticles when used in a nanoprecipitation process as described below.

Thus, an exemplary embodiment of the disclosed nanoparticle system is entirely comprised of light-activatable prodrug monomers of the chemotherapy agent doxorubicin. Exemplary monomers of DOX-PCB are capable of self-assembling into nanoparticles, due to the dual hydrophobic/hydrophilic nature of the molecule, upon injection into aqueous solution using a single-step nanoprecipitation process. The nanoprecipitation process can be used to produce the desired compact structure of the nanoparticle which entirely or substantially contains prodrug monomers, and which is configured to have a hydrophobic core and a hydrophilic surface.

A schematic of the exemplary nanoparticle is shown in FIG. 3. The hydrophilic and hydrophobic regions of the monomer in its hairpin conformation are color-coded in black and gray. When nanoprecipitated, the monomers form a spherical cluster comprising the core of the nanoparticle while the monomers at the surface point their hydrophilic tails into the water. The exemplary DOX-PCB nanoparticle comprises DOX-PCB monomers on the outer layer of the nanoparticle oriented themselves such that the PEG chain interfaced with the water and prevented hydrophobic interactions between nanoparticles and subsequent aggregation. The surface of the nanoparticle is coated with the biotin-terminated PEG tail.

These nanoparticles are formed using only the light-activatable prodrug monomers without the need for any support scaffold material, thereby maximizing the payload capacity by utilizing the entire volume of the nanoparticle to carry the prodrug. This is different from most other nanoparticle designs that require a scaffolding or structural barrier to contain the drug payload. These structural components take up valuable volume inside the nanoparticle which reduces the amount of payload that can be loaded. In certain implementations, the DOX-PCB nanoparticles can have a loading efficiency of the prodrug of at least 95%, preferably about 99%, as determined by high pressure liquid chromatography showing very low quantities, such as less than 5% or less than 2%, of DOX-PCB left in the supernatant after the nanoparticles are removed from the solution through filtration.

In some examples of the disclosed technology, the prodrug molecule, DOX-PCB, can fold over on itself to form a hairpin conformation that allows it to be nanoprecipitated into self-assembled nanoparticles of pure prodrug. Upon light exposure, DOX-PCB releases active doxorubicin (DOX) which is free to subsequently penetrate into cells. This is distinct from other particles or particle systems where only pure drugs or biochemically activated prodrugs are loaded into preexisting nanoparticle scaffold structures. This is also distinct from other nanoparticle structures that, upon light exposure, release only free radicals, singlet oxygen, or high energy compounds which have no cell specificity in their action and are not capable of the same cellular penetration as traditional therapeutic compounds.

B. Delivery of Nanoparticles

In some implementations, the nanoparticles are administered systemically via injection, infusion or ingestion. For example, the nanoparticles administered through these administration routes enter the body circulation system, e.g., blood stream, to reach different parts or regions of the body. For example, the size and shape of the nanoparticles enable extravasation through the leaky vasculature of the tumor allowing them to accumulate in the tumor region. This helps reduce the prodrug accumulation in healthy tissue while allowing it to preferentially accumulate in the tumor. The size and shape of the nanoparticles also allow for cellular internalization in the tumor region. The nanoparticles can also accumulate unintentionally in healthy tissue, but will not be light-activated in these locations. Here, the prodrug form of the drug will greatly reduce the local toxicity in healthy tissue, thereby reducing dose limiting side effects.

The disclosed delivery system includes nanoparticles configured to have a shape, size and various possible surface coatings that allow the nanoparticles to circulate for longer periods of time, e.g., as compared to prodrug monomer itself injected freely without a carrier into circulation. For example, a free produg configuration can result in a change in pharmacokinetics and biodistribution of the drug. The shape and size of the nanoparticles of the disclosed technology allow them to preferentially extravasate and accumulate into the tumor tissue by way of the tumor's “leaky” vasculature in a manner distinct from the free monomer and other systems. For example, longer circulation time of the nanoparticle formulation of the light activatable prodrug is important in certain applications because it allows more of the prodrug nanoparticles to pass through and preferentially accumulate in the desired tissue region than the shorter circulation time of the monomer allows. This ultimately leads to a larger delivered dose than for the free monomers having a short circulation half-life.

The nanoparticle formulation of the disclosed technology does not require a solubilizing vehicle that is required to solubilize some of the pure monomers for injection reducing any additional side effects from the solubilizing vehicle. Since the nanoparticle is composed of many prodrug monomers and capable of being internalized by cells in particle form, the nanoparticle provides the capability to internalize and activate many monomers in a short amount of time inside a cell.

Also for example, in some implementations, the disclosed nanoparticle design can be configured such that the drugs that are released from the prodrug nanoparticle are chemotherapy agents. Such chemotherapy agents can have a level of tumor specificity in their therapeutic mechanism so they can be activated next to healthy tissue with far less toxic effect than the production of free radicals. They do not create indiscriminate cell damage, but rather they have very specific therapeutic activity and can specifically accumulate in the cell. This allows the active agents to travel further than, for example, free radicals, and also allows the activation of the prodrug to occur within the nanoparticle itself because the active agents can leave without being consumed. An example of this is the release of doxorubicin which can easily pass through a cell membrane and accumulate in the cell nucleus where it blocks normal DNA manipulation. In the described design, the prodrug monomers remain in nanoparticle conformation due to hydrophobic interactions. The disclosed system can be configured so that the particles do not require any external encapsulating structure.

In some examples, the prodrug nanoparticles containing a chemotherapy prodrug can be administered systemically after surgical resection of a tumor, allowing the surgeon to light activate the nanoparticles in the localized regions where tumor margins might have been left behind. This would allow for the treatment of any cancer cells that may have been left behind (which can be particularly aggressive in the tumor margin region) or any tumor cells dislodged or loosened from the physical impact of the surgery. Even if all of the primary tumor cells were successfully removed by the surgeon, the surgical wound site can still be a prime environment for metastatic or circulating tumor cells to settle and colonize. Thus, prodrug activation in the wound site post-surgery can potentially prevent metastasis or cancer recurrence by creating a high concentration of chemotherapy agent at the target site. As an additional application, the prodrug nanoparticles can be administered systemically prior to, during, or just after needle biopsy of a tumor and then light-activated along the needle tract. Light application can include but is not limited to a fiber optic light source attached to the biopsy needle. Similar to the post-surgery application, this can treat cancer cells loosened or dislodged by the needle and help to prevent metastasis development in the wound healing environment along the needle tract.

In some examples, the administration and light activation of the nanoparticles in the tumor can be done before surgical resection to help kill cells in the tumor margins and throughout the tumor to reduce the chances of the surgical procedure dislodging and releasing potentially metastatic tumor cells into circulation.

C. Light-Activation of Nanoparticles

Currently, the ability to inject systemically circulating compounds but restrict active drug exposure to predefined regions of tissue is not achievable using other nanoparticle platforms due to uncontrolled accumulation of active agents in healthy tissue. Implementations of the exemplary delivery platform provide several features and advantages that allow physicians to restrict exposure of active drugs to predefined localized regions of tissue within the body that are selectively exposed to the activating light.

Some existing systems, for example, include nanoparticles that carry prodrugs that are activated by the cellular environment and enzymatic activity. The disclosed technology uses light activation mechanism of prodrugs that can be spatially controlled to a much higher degree, e.g., as compared to metabolic or enzymatic controls. For example, with metabolic activation, the prodrug can be converted to the active drug in any tissue or cell that has similar enzymes and/or biochemistry as the target tissue. This is particularly a problem for the liver which has a much higher degree of enzymatic activity than most other tissue and potentially can cause massive activation of the prodrug leading to local toxicity. The disclosed light activation mechanism will only occur at significant levels within the tissue that is preferentially exposed to the light but not in other unintended tissues. This limits the active drug exposure of tissues outside the region where the light is preferentially delivered.

The light-activation mechanism of the disclosed technology includes several advantages, e.g., safety of delivery and efficiency in release of the desired chemical substance to the subject. For example, this is in contrast to a gamma ray triggered release mechanism. Gamma rays are not capable of being preferentially absorbed by chemical bonds as is possible with wavelengths, e.g. in the UV and visible spectrum. Wavelengths that are preferentially absorbed by chemical bonds enable consistent drug activation and/or release from the prodrug. Therefore, gamma rays are not efficient at activating photo-triggered chemicals.

As an example, in the case of chemotherapeutic agents, the disclosed system allows doctors to limit the systemic exposure of active chemotherapy agents to healthy tissue while achieving a concentrated dose of the active drug within the tumor tissue that is exposed to the light. The light can be delivered to the tumor tissue using fiber-optic and light emitting diode technology. Implementations of the described systems and techniques will reduce the systemic side effects experienced with chemotherapy and other drugs. The disclosed nanoparticle delivery technology can also be applied to reduce the systemic side effects resulting from the treatment of other medical conditions where a localized action is required and systemic side effects result from uncontrolled drug exposure.

In an exemplary embodiment, pure doxorubicin in its active form is activated and released from the nanoparticles only when exposed to 365 nm light. Ideally, the internal tissue has low absorption of the light of the triggering wavelength and the light does not penetrate significantly through the skin of the subject being treated, e.g., less than 1 mm through human skin. This ensures that the only significant amount of triggering light present in the body is that delivered to the targeted region. The localized delivery of light of the triggering wavelength can be achieved by using a custom-designed system based on LED/fiber-optic system known in the art.

For example, 365 nm light is an important wavelength for activation because it has sufficient penetration depth through at least 1 cm of tumor tissue to allow for activation of the DOX-PCB monomer while having significant absorption by melanin in the skin to block external sources of 365 nm light from causing uncontrolled activation of the prodrug in internal tissues. Higher intensities or longer exposure times of 365 nm light could cause activation in even larger tissue volumes allowing the light exposure to be tuned to a desired tissue volume.

The activating wavelength or wavelengths of light that activate the drug from the prodrug monomers can be delivered to the desired tissue region using fiber optic and/or light emitting diode technology. The light emitting diodes can be made very small and can be implanted into many tissue regions of the body. They can be controlled from the surface of the body to turn on and off and to adjust the level of light intensity. This allows the physician to inject or infuse the DOX-PCB nanoparticles into circulation and then select when the activating light will be turned on in the desired tissue region. For example, the activating light can also be delivered endoscopically or during a needle biopsy by coupling a fiber optic or light emitting diode with an endoscope or biopsy needle.

The exemplary implementations of the exemplary DOX-PCB drug delivery system included the following exemplary results. The DOX-PCB monomer has been successfully injected into circulation in mice where it has been demonstrated that the light-exposed tumor preferentially accumulated active DOX. These results demonstrate that light can be delivered to the tumor region using a fiber-optic/light-emitting-diode system to preferentially activate the prodrug.

In some examples, the light emitting diode delivery system can have many different forms including, for example, single diodes; or networks of diodes, e.g., (i) in line with one another, (ii) branched structures, (iii) distributed over multi-dimensional meshworks that can wrap around internal organs, and/or (iv) catheter based.

The non-activated prodrug nanoparticles of DOX-PCB have a cellular toxicity that is 30 times less than pure doxorubicin. Exemplary cellular localization studies were performed using the exemplary light-activatable prodrug monomers of doxorubicin, which showed that the prodrug carried by the nanoparticles passed into the cell cytoplasm but did not intercalate into the DNA like pure doxorubicin. For example, exposure to 365 nm light increased the toxicity of the nanoparticle sample to A549 human lung cancer cells by releasing pure doxorubicin which demonstrated normal DNA intercalation capabilities.

FIG. 4 shows a schematic representation of the exemplary concept of nanoparticle delivery of light-activatable prodrug followed by light activation in a localized tissue region (e.g., a tumor). The schematic diagram of FIG. 4 shows the overall concept that uses the nanoparticle formulation of the light-activated prodrug to precisely deliver the active drug to the desired tissue region, such as a tumor. For example, the nanoparticles can be administered systemically via intravenous injection or infusion. The activating light can then be delivered specifically to that tumor region using current light emitting diode and fiber optic technology. The prodrug only converts to the fully active therapeutic form when exposed to the delivered light. The light exposure keeps the activation region spatially localized reducing wide systemic exposure.

In some examples, an additional fiber optic detector system or detector-based system can be employed in combination with the light delivering light emitting diode or fiber-optic system. This detection system can look for fluorescence from the particle or light absorption changes that indicate the presence and quantity of the nanoparticles in the immediate vicinity of the activating system. This can confirm the presence of the drug delivery nanoparticles. The detection system can also confirm the light activation of the prodrug in the nanoparticles by looking for absorption or fluorescence shifts. This information can help the physician decide when the best time or times are to turn on the activation system or to determine the successful delivery of active drug to the tissue region. It can also be used to help make sure the entire system is located in the correct tissue region based on local tissue light absorption information. It could also help determine resulting changes to the immediate tissue composition (such as if the tumor is shrinking and being replaced by healthy tissue). This added functionality would allow both therapy and diagnostics to occur at the same time (theranostics).

D. Advantages of the Disclosed Nanoparticles

Generally, the conventional thinking about nanoparticle drug delivery vehicles is to design their structures to result in a very slow release rate of the pure drug payload. The idea is that this slow release rate will limit the amount of drug that leaks out and acts on the healthy tissue during circulation. Over time the particles will accumulate in the desired tissue region. The accumulated particles continue to slowly release drug into the desired tissue region resulting in therapeutic effects over time. However, there are several main problems with such conventional approaches.

(1.) One problem with conventional systems is the accumulation of the majority of the nanoparticles in healthy tissue. Only a fraction of the injected nanoparticle dose ever reaches the desired tissue region, such as a tumor. The rest circulates throughout the body where it accumulates in various healthy tissues. For example, the majority accumulates in the liver and spleen as these organs perform their normal clearance functions. The problem comes from these particles continuing to slowly leak their drug contents into the healthy tissue where they have accumulated. This can lead to local toxicity of the healthy tissues and creates dose-limiting side effects experienced by patients. Active targeting agents such as antibodies or peptides may be added to nanoparticles to increase the fraction that accumulates in a desired tissue, but it is still impossible to prevent significant accumulation of these nanoparticles in healthy tissue especially considering the clearing functions performed by the liver and spleen.

(2.) A second issue stems from the slow release of drug from the vehicles. The drug clears from the tumor tissue over time which means that the slow release of drug from the nanoparticles limits the dose of bioavailable drug that can be achieved in the tumor tissue. These low leak rates prevent the drug from achieving its maximum therapeutic potential in the tumor tissue.

(3.) Another problem in conventional systems is a need for structural scaffolding. The structural scaffolding needed in other nanoparticle designs can take up valuable space within the vehicle that could be better used for drug payload. The presence of the scaffolding requires more nanoparticles to be injected and more nanoparticles to accumulate in the desired tissue region to have a therapeutic effect.

(4.) Another problem in conventional systems is that existing chemotherapy nanoparticles that do employ prodrug activation schemes rely on biochemical activation of the prodrug to release active drug. These biochemical processes can also be present in other places within the body outside the desired tissue region, especially in the liver, resulting in uncontrolled release into healthy tissue.

The disclosed technology addresses these four and other problems directly.

For example, the disclosed nanoparticle design directly addresses the issue of the nanoparticles unintentionally accumulating in non-target tissues because the payload of the exemplary nanoparticles is entirely in the form of inactive prodrug. No active drug is contained within the exemplary nanoparticles of the disclosed technology when injected. The prodrug toxicity is greatly reduced from that of pure drug. The accumulation of the disclosed nanoparticles comprising less toxic prodrugs in healthy non-target tissues results in greatly reduced side effects. For example, the tumor tissue, or other tissues where active drug is required, is selectively exposed to the activating wavelength of light that triggers the chemical conversion of the prodrug into the active drug. This allows the prodrug nanoparticles to release pure drug inside the target tissue, but nowhere else in the body where they might unintentionally accumulate. This reduces the dose-limiting toxicities experienced by other nanoparticle drug delivery systems. For example:

I. The light can be preferentially delivered to the desired tissue region using fiber optic and light emitting diode technology that can be implantable or endoscopic. The wavelength of light has been chosen to prevent widespread activation of the prodrug from external sources allowing the delivered light to be the sole activating trigger.
II. The prodrug itself is resistant to metabolic activation. This is different from other traditional prodrug designs that have relied heavily on biochemical activation schemes, e.g., such as activation by enzymatic activity. The use of light as the activating trigger avoids the normal metabolic activity of the healthy tissue, especially the liver, activating the prodrug in healthy tissue.

The disclosed nanoparticle design directly addresses the issue of slow release of active payload from the nanoparticle by using the light activation mechanism to convert the prodrug monomers to active drug at a fast rate. Since the nanoparticle is not designed to hinder the outward release of active drug, the activated drug can quickly become bioavailable. Unlike many traditional drug delivery nanoparticle designs, no scaffold is required to degrade in order to release the payload from the exemplary particles. This design achieves the maximum possible dose in the tumor tissue. Additionally, the fast burst release of drug from the exemplary nanoparticle of the disclosed drug delivery systems upon exposure to triggering light has the potential to be more effective on drug-resistant cells than traditional nanoparticle designs. For example, one of the major mechanisms involved in chemotherapy drug resistance of cancer cells is the presence of drug efflux pumps in the cell membrane which pump out the chemotherapy agent. By activating a fast burst release of drug from internalized nanoparticles or prodrug monomers inside the cell membrane, the disclosed system has the potential to overwhelm the efflux pumps and create a therapeutic dose of drug inside the cell before it can be pumped out.

Also, for example, the nanoparticles of the disclosed technology directly address the payload capacity issues experienced with other conventional nanoparticle designs that require structural scaffolding. A key aspect of nanoparticle design of the disclosed technology is that the core, or the entire particle itself, is substantially comprised of prodrug. The nanoparticle achieves the maximum therapeutic load possible since it is substantially composed of prodrug or has a core substantially composed of prodrug that does not require any extra space-consuming structural scaffold material. This allows more payload to be delivered per nanoparticle than polymer scaffold-based nanoparticle designs that are the same size. Additionally, since the disclosed nanoparticles can be formed from precipitating the prodrug material rather than encapsulating the prodrug within a matrix or scaffold, such designs of the disclosed technology avoid drug loss due to low encapsulation efficiency, e.g., during manufacturing, which is a common issue for polymer-based particles.

The nanoparticle designs of the disclosed technology directly address the lack of tissue specificity for biochemical prodrug activation schemes. Implementation of the disclosed systems include techniques that use light to activate the prodrugs in the nanoparticles, e.g., which bypasses the issue of lack of distinction between tumor and healthy tissue biochemistry. Such techniques also avoid problems associated with massive unwanted prodrug activation in the liver.

In sum, conventional nanoparticles that carry active drug, e.g., including biodegradable scaffolds and liposome based nanoparticles, may unavoidably be delivered to healthy tissue where the nanoparticles accumulate, including the liver and the spleen, e.g. regardless of the targeting mechanism and regardless of the biodegradation scheme. The disclosed nanoparticle is different because the light activation mechanism of the prodrug payload confines the active drug preferentially to the tissue where the activating light is delivered. The disclosed nanoparticle does not require any structural scaffolding and can carry a higher amount of payload than a traditional scaffold-based nanoparticle of the same size. Additionally, the disclosed nanoparticle does not require degradation of a biodegradable structure in order to release drug and is designed to retain the drug better than polymer scaffold-based particles during circulation but still release active drug in the tumor when triggered by exposure to the activating wavelength of light. The systems of the disclosed technology can be configured to produce a release rate of active drug from the photoactivatable nanoparticles more rapid than what is possible by existing systems, e.g., such as polymer scaffold-based nanoparticles, thus enabling the disclosed system to achieve higher concentrations of the active drug in the tissue over a given time period.

In some implementations, the disclosed nanoparticle compound can be structured as a photocleavable prodrug nanoparticle composed of monomers of photocleavable doxorubicin prodrug, e.g., referred to as DOX-PCB. It is noted that Doxil® is a currently available pegylated liposome-encapsulated form of doxorubicin (DOX). Both Doxil® and the disclosed prodrug nanoparticle formulation ultimately result in the accumulation of pure doxorubicin in the tumor tissue. However, an important, overall difference between these two particles is that the prodrug nanoparticles described here can perform similar anti-tumor functions as Doxil® without the high degree of toxic dose-limiting side effects. For example, in addition to the tumor, Doxil® unintentionally accumulates in the bone marrow causing neutropenia and in the creases of the hands and feet causing ulcer formation. These side effects result from Doxil® releasing active drug payload into these tissue regions. In contrast, the disclosed nanoparticles effectively treat the tumor while greatly reducing or eliminating toxicity to the user/patient, e.g., since the exemplary particles of the disclosed technology carries only the prodrug of DOX, not active DOX. For example, in exemplary implementations of the exemplary nanoparticle formed of DOX-PCB, the prodrug nanoparticles are at least 20 times, at least 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, or at least 200 times less toxic than the pure DOX. In other embodiments, the prodrug nanoparticles are about 20 times, about 30 times, about 40 times, about 50 times, about 60 times, about 70 times, about 80 times, about 90 times, about 100 times, about 110 times, about 120 times, about 130 times, about 140 times, about 150 times, about 160 times, about 170 times, about 180 times, about 190 times, or about 200 times less toxic than the pure DOX. For example, the exemplary particle having the prodrug can accumulate in the same places within the body as Doxil®, including the tumor. However, the prodrug nanoparticles that accumulate in healthy tissue, e.g., such as the bone marrow, do not receive the activation dose of light that is delivered specifically to the tumor tissue and thus do not release pure DOX into the bone marrow and other healthy tissues where the activation light does not reach. This limits the exposure of tissues outside the desired location to active DOX.

In some implementations of the disclosed technology, the disclosed drug delivery system can include the following exemplary features or be implemented in the following exemplary ways.

In one example, a therapeutic drug delivery nanoparticle is substantially comprised of photoactivatable prodrug monomers which upon exposure to an activating wavelength of light release a subcomponent of the monomer, which subsequently acts upon specific biological targets or has specific therapeutic modes of action.

For example, the monomers of the exemplary therapeutic drug delivery nanoparticle can be substantially comprised of hydrophobic and hydrophilic regions and/or other regions that possess the necessary chemical nature to allow the monomers to self-assemble into nanoparticle form.

For example, the monomers of the exemplary therapeutic drug delivery nanoparticle can have different chemical forms, e.g., all of which lend the necessary chemical nature to allow the different monomers to self-assemble together into nanoparticle form so that the nanoparticles are substantially comprised of the different monomer types.

For example, the monomers of the exemplary therapeutic drug delivery nanoparticle can have different chemical forms which are efficiently activated by different light wavelengths allowing temporal control over the release of different therapeutics from the different monomers inside the particle.

For example, the monomers of the exemplary therapeutic drug delivery nanoparticle can have different chemical forms which are efficiently activated by the same light wavelengths allowing different therapeutics to be released from the nanoparticle at the same time.

For example, the monomers of the exemplary therapeutic drug delivery nanoparticle can possess the necessary chemical nature to allow the monomers to self-assemble into nanoparticle form and create a surface on the nanoparticle that increases circulation time of the nanoparticle.

For example, the monomers of the exemplary therapeutic drug delivery nanoparticle can allow the nanoparticle surface to be coated by additional surface coatings to change the surface properties of the nanoparticles.

For example, the monomers of the exemplary therapeutic drug delivery nanoparticle can allow targeting ligands to extend from the nanoparticle surface.

For example, the monomers of the exemplary therapeutic drug delivery nanoparticle can allow imaging contrast agents to be incorporated into the nanoparticle or on its surface.

In some examples, a light delivery system is substantially comprised of light emitting diodes which can be implanted or positioned into or within the proximity of the desired tissue region of the body where they are controlled to emit light at the wavelengths which will result in activation of the photoactivatable prodrug monomers of the exemplary therapeutic drug delivery nanoparticle.

For example, the exemplary light emitting diode system can be configured such that the individual light emitting diode elements are arranged in, but not limited to, networks that resemble, for example, nets, sheets, or strings that allow for light exposure to desired tissue regions.

For example, the exemplary light emitting diode system can be configured such that the light emitting diode elements or arrays are employed in combination with an additional fiber optic detector system or detector-based system that are designed to determine the presence and quantity of the exemplary drug delivery nanoparticle, e.g., using, but not limited to, fluorescence or light absorption changes caused by the nanoparticles.

For example, the use of the exemplary light detection system can help determine proper placement of the system in the desired tissue region through light absorption measurements.

For example, the use of the exemplary light detection system can help measure the concentration of the exemplary drug delivery nanoparticles in a desired tissue region to determine the correct temporal activation of the prodrug monomers.

In some examples, the use of focused ultrasound is directed to the tissue region of interest to create capillary and micro-capillary damage to enhance the retention of the exemplary nanoparticles in the tissue of interest.

For example, the enhancement for the tissue retention of the exemplary nanoparticles can be implemented such that the focused ultrasound is combined with injectable microbubbles that circulate into the tissue region of interest.

Implementations of the subject matter and the functional operations described in this patent document are illustrated in the working examples below.

### Example 1

### DOX-PCB Nanoparticle Synthesis

Doxorubicin hydrochloride (DOX) was purchased from Qventas (Branford, Conn.) and Sigma (St. Louis, Mo.). Water soluble photocleavable biotin-NHS (PCB) was purchased from Ambergen (Watertown, Mass.). High pressure liquid chromatography (HPLC) grade acetonitrile was purchased from Fisher Scientific (Fairlawn, N.J.).

The DOX-PCB monomers were synthesized using the procedure described in Ibsen et al. See Ibsen et al., Pharm. Res. 27: 1848-1860 (2010). To form nanoparticles from the DOX-PCB prodrug, a single-step nanoprecipitation method was used. The DOX-PCB monomer was first dissolved in acetonitrile at a concentration of 0.1 mg/ml. A 168 μl volume of this solution was aspirated into a 1 ml syringe fitted with a 30 gauge 1 inch needle, and then added dropwise to 2 ml of ultrapure water under vortex. The hydrophobic ends of the prodrug molecules clustered together to form particles in order to minimize contact with the water. The resulting sample was stirred gently for 48 hours at room temperature while open to the atmosphere to evaporate the acetonitrile. This evaporation was performed to help ensure the stability of the nanoparticles and the biocompatibility of the sample. The sample was protected from ambient light during the stirring process.

The particles were then concentrated using a centrifugal evaporator to remove the desired amount of water. The particles did not aggregate as they were concentrated under the centrifugal force. Care was taken to ensure the evaporation rate did not result in the freezing of the water. DOX-PCB concentration was measured through light absorption using a Nanodrop ND-1000 spectrophotometer (Thermo Fisher Scientific, Waltham, Mass.). All DOX-PCB nanoparticle solution concentrations are given in terms of DOX-PCB molecule content.

This method can produce nanoparticles that are measured by scanning electron microscopy (SEM) and dynamic light scattering to be in the size range of 40-150 nm in diameter. It is noted, for example, that these exemplary particles are stable in aqueous solution at 4° C. for several months, e.g., for over 6 months, while covered with foil for light protection. The nanoprecipitation methods can be easily scaled up for mass production of the particles. An example of a scaled up nanoprecipitation process is the use of continuous nanoprecipitation by using multi-inlet vortex reactors or flow channels. Nanoprecipitation is just one method exemplary to produce self-assembled nanoparticles. The monomers can be designed to use other methods to cause the self-assembled formation of nanoparticles from the monomers.

The nanoparticles described here had >95% prodrug entrapment since no significant amount of DOX-PCB monomer was found in the water phase after the particles had been removed with filtration. The drug content (drug loading) of DOX in the particle is 45% w/w. The nanoparticles were able to be concentrated to at least 0.22 mg of DOX-PCB per mL without aggregation supporting that the monomers on the outer layer of the nanoparticles most likely oriented themselves such that the PEG chain interfaced with the water and prevented hydrophobic interactions between the nanoparticles and subsequent aggregation. The surface coating of the nanoparticles with the biotin-terminated PEG tail was confirmed by fluorescence imaging to show binding of the nanoparticles to the surface of avidin coated polystyrene beads where bare polystyrene beads showed no binding. High concentrations in combination with the prodrug nanoparticles' lower toxicity could potentially allow for smaller infusion volumes to be achieved or could even allow for IV injection.

### Example 2

### Particle Characterization

The DOX-PCB nanoparticles were characterized using three different methods. The size distribution of the particles was first evaluated by the Nanoparticle Tracking Analysis (NTA) technique using a NanoSight LM10 system (NanoSight Ltd., Amesbury, UK) at room temperature. Two samples of 300 μl volume were introduced into the viewing unit for the particles to be tracked and sized on a particle-by-particle basis using the NTA technique. Size is reported as the average of the peak value of the size distribution from each sample. A video clip of the particles' Brownian motion was also acquired using the NanoSight system.

The size results acquired by NTA were confirmed using a Zetasizer Nano-ZS dynamic light scattering (DLS) system (Malvern Instruments, Worcestershire, UK) at room temperature with backscattering angle of 173°. Size measurements were obtained in triplicate along with polydispersity indices. Zeta potential measurements were also acquired in triplicate using this instrument.

Finally the surface and shape morphology of the nanoparticles were evaluated using scanning electron microscopy (SEM) with a Phillips XL30 SEM system. The manufactured nanoparticle sample was diluted on the order of 10,000 times from its original concentration to allow observation of individual particles. A 2 μl sample of the diluted sample was placed on the surface of a polished silicon wafer fragment and allowed to dry at room temperature overnight. The sample was coated with chromium and then imaged.

More specifically, the size results obtained from Dynamic Light Scattering (DLS) showed that the DOX-PCB nanoparticles had a unimodal size distribution with an average diameter of 102.5±1.6 nm as shown in Table 1. The size results obtained from Nanoparticle Tracking Analysis (NTA, Table 1) confirmed the nanoparticle size at around 100 nm in diameter. Zeta potential was measured at −41.6±3.6 mV, which supports that the nanoparticles have sufficient repulsive interactions to be stably monodispersed in solution.

TABLE 1
Characterization of DOX-PCB nanoparticle size and
zeta potential (mean ± standard deviation)

| Diameter | | Diameter | Zeta |
|---|---|---|---|
| by DLS | Polydispersity | by | Potential |
| (nm) | Index | NTA (nm) | (mV) |
| 102.5 ± 1.6 | 0.120 ± 0.012 | 96.5 ± 6.4 | −41.6 ± 3.6 |

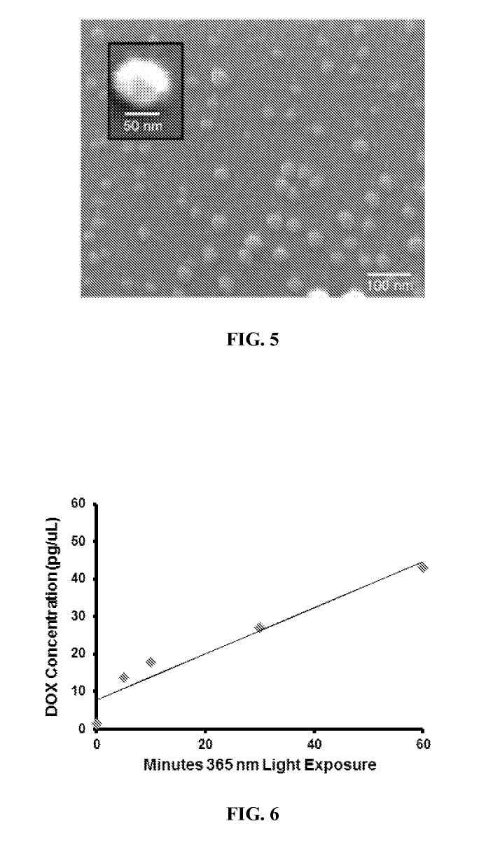

Scanning electron microscope images of the particles are shown in FIG. 5. The particles are spherical in shape with a size range that appears slightly smaller than the results obtained by DLS and NTA, which may be an effect of the nanoparticle drying preparation used for SEM.

### Example 3

### Light Activated Drug Release Characterization

The nanoparticle samples were evaluated for their release of pure DOX after receiving various amounts of 365 nm light exposure. Samples of 10 μl of DOX-PCB nanoparticles at 31 μM were placed into individual wells in a 96-well flat bottom assay plate (BD Biosciences, San Jose, Calif.) with opaque black walls and a clear bottom. The black walls helped to prevent reflections of 365 nm light inside the well and ensure uniformity of exposure between samples. The clear lid of the plate was fitted on top to help reduce the evaporation rate of the water. The samples were exposed to 2.3 mW/cm² of light from a Mercury Short Arc HBO bulb from OSRAM (Munchen, Germany) with a 330-380 nm bandpass filter for increasing durations of time from 0 to 60 min. This measured light intensity took into account the absorption of 365 nm light by the lid. The samples were then collected and their volumes adjusted with water to 10 μl if any evaporation occurred.

In an in vivo application, the DOX that was released from the nanoparticles would immediately diffuse away from the particle and associate with plasma proteins, cellular proteins, and DNA. In the specific case of this experiment the DOX-PCB nanoparticles were suspended at a high concentration in pure water which could allow the free DOX released from the particles to immediately associate with other particles. To help prevent DOX from being sequestered into other particles, the 365 nm light exposed samples were then mixed with dimethyl sulfoxide (DMSO) in a 0.2/1 v/v ratio. The samples were then bath sonicated for 5 minutes. The presence of DMSO in the sample helped to keep the released DOX in solution so that it could be quantified by LC-MS/MS.

The DOX content of the samples was then quantified by LC-MS/MS using an Agilent 1260 liquid chromatograph (LC) system coupled with a Thermo LCQdeca mass spectrometer (MS). The LC-MS/MS analysis used positive ion mode electrospray ionization (ESI) as the ion source with source voltage of 5 kV, capillary temperature of 250° C., auxiliary gas flow rate of 20 units, and sheath gas flow rate of 80 units. A CAPCELL MG III C-18 column (Catalog number 92744, ID 2.0 mm×length 50 mm, particle size 3 μm) was used (with guard column) for LC separation. The mobile phase A was 5% methanol in water with 0.1% formic acid. The mobile phase B was pure methanol with 0.1% formic acid. The LC gradient was increased from 30% mobile phase B to 95% mobile phase B in a duration of 10 minutes, then held at 95% B for 5 minutes, brought back to 30% B in 1 minute, and then held at 30% B for 6 minutes. The LC flow rate was held at 0.20 ml/min. Using these LC conditions, the DOX and the two isomers of DOX-PCB were separately eluted from the LC column with a retention time of about 9.8 minutes for DOX, and about 13.2 and 13.7 minutes for the two isomers of DOX-PCB. DOX had a molecular ion peak at m/z 544 ([M+H]⁺). Under positive ion mode ESI-MS/MS analysis, a major fragment peak of DOX was seen at m/z 396.8 with a normalized collision energy of 30%. Both of the DOX-PCB isomers had molecular ion peaks at m/z 1244.4 ([M+Na]⁺). Under positive ion mode, the ESI-MS/MS analysis showed a major fragment peak ([M+Na]⁺) at m/z 848.3 with a normalized collision energy of 35%. Selected reaction monitoring (SRM) mode was used to acquire the m/z 396.8 fragment ion peak which were used for quantification of the DOX.

Pure DOX was activated and released from the nanoparticles in a linear relation with the amount of 365 nm light exposure (R²=0.93) as shown in FIG. 6. A higher dose of 365 nm light, either in intensity or duration, would result in higher amounts of active DOX.

### Example 4

### Cellular Localization

The intracellular localization of the DOX-PCB nanoparticles and pure DOX was studied using the PTK2 epithelial cell line. These cells were used because they are susceptible to DOX and remain flat during mitosis, allowing for enhanced visualization of the nuclear region. This visualization capability was important since one of the main therapeutic modes of action for DOX is through DNA intercalation. The DOX-PCB nanoparticles and pure DOX are both naturally fluorescent which allowed them to be easily tracked through the cell. The PTK2 cells were plated in glass bottom petri dishes (MatTek Corporation, Ashland, Mass.) and were incubated in advanced MEM Media from Gibco (Invitrogen, Grand Island, N.Y.) with 2% fetal bovine serum, nonessential amino acids, 110 mg/L sodium pyruvate, and without penicillin-streptomycin or L-Glutamine.

A 1 ml media solution of DOX-PCB nanoparticles was prepared by first adding 10×DPBS to a 4.1 μM solution of DOX-PCB nanoparticles in water to achieve a 1×DPBS concentration, and then adding the resulting solution to a phenol-red free formulation of the advanced MEM media described above to achieve a final DOX-PCB content of 2 μM. This spiked media sample was then added to the glass bottom petri dish along with PTK2 cells seeded which had the previous incubation media removed. A 1 ml media solution of 2 μM DOX was prepared in the same manner with the DPBS content matched to the above nanoparticle solution, and the DOX media solution was exposed to the cells in the same manner as described above. The cells were incubated with the spiked media samples for 2 hours. Live fluorescent images of the cells were obtained using a Zeiss Axiovert 200 M Microscope (Zeiss, Thornwood, N.Y.) using an HCredl rhodamine filter cube from Chroma (Rockingham, Vt., USA) and a 63× phase III, NA 1.4 oil immersion objective. All microscope control and imaging utilized the RoboLase system.

To study the change in cellular localization after activation, the cells incubated with the DOX-PCB nanoparticles were exposed to the 365 nm light source for 60 seconds. The cells were then allowed to incubate in the 365 nm light-exposed media for 1 hour. The cells were reanalyzed using the microscopy system described above. The RoboLase system allowed the same cells that where imaged before 365 nm light exposure to be relocated and imaged again after 365 nm light exposure.

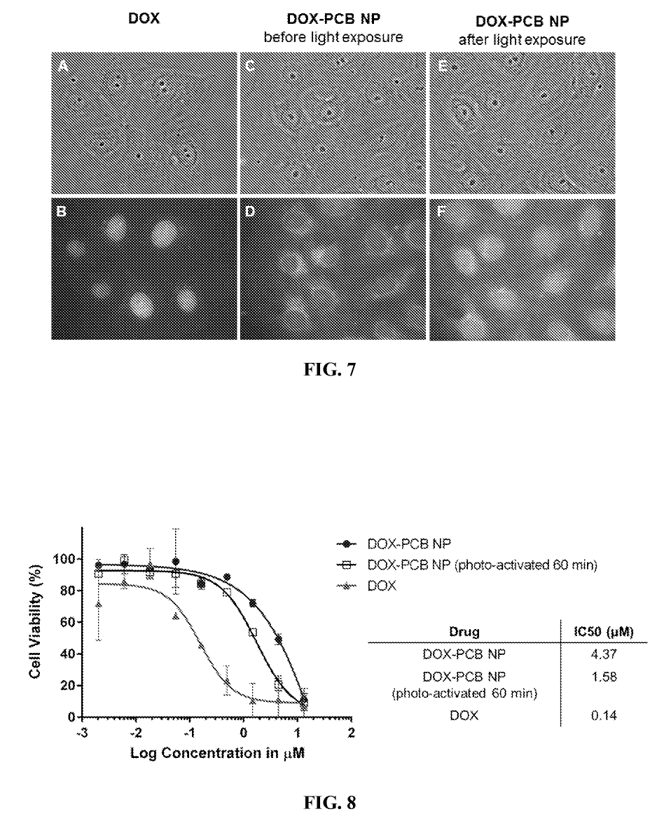

The localization of free DOX and the DOX-PCB nanoparticles in the PTK2 kidney epithelial cell line is shown in FIG. 7. Both DOX and DOX-PCB are inherently fluorescent allowing them to be tracked within a cell. The PTK2 cells were chosen due to their unique tendency to remain flat during replication improving the ability to image the nuclear region and determine cellular localization. One of the known modes of therapeutic action of pure DOX is as a DNA intercalator and it has also been observed to have therapeutic effect through interactions with DNA polymerase I and topoisomerase II. Free DOX is seen to strongly associate with the cell nuclei as shown in panel 7B. Prior to 365 nm light exposure, the DOX-PCB in the nanoparticles can be seen in the cell cytoplasm possibly binding to the Golgi apparatus or the endoplasmic reticulum, but not associating with the cell nuclei. After exposure to the 365 nm light, the nuclei of the cells show increased fluorescence supporting that the DOX released from the DOX-PCB was associating with the nucleus as therapeutically expected.

### Example 5

### Cytotoxicity

The human lung cancer cell line A549 was purchased from the American Type Culture Collection (Manassas, Va.). Dulbecco's Modified Eagle Medium (DMEM) cell culture media and trypsin-EDTA (ethylenediaminetetraacetic acid) for cell culture were purchased from Mediatech, Inc. (Manassas, Va.). The penicillin-streptomycin used as a media supplement and the DMEM media without phenol red were purchased from Gibco (Invitrogen, Grand Island, N.Y.). Fetal bovine serum used as a supplement in the DMEM media was purchased from Hyclone (Logan, Utah). Dulbecco's phosphate buffered saline (DPBS) was purchased from Hyclone Laboratories Inc. (Logan, Utah). All water was purified using the Milli-Q purification system from Millipore Corporation (Billerica, Mass.). XTT was purchased from Sigma (St. Louis, Mo.).

An IC50 (e.g., drug concentration at which cellular viability was reduced by 50%) study was conducted to determine the toxicity of the DOX-PCB nanoparticles before and after 365 nm light exposure relative to free DOX using the human lung cancer cell line A549 which was purchased from the American Type Culture Collection (ATCC) (Manassas, Va., USA). The cells were grown on sodium pyruvate-free DMEM media containing L-glutamine, 4.5 g/L of glucose, penicillin-streptomycin and 10% Fetal Bovine Serum. The adherent cells in the expansion flask were detached using Trypsin (0.25% T/2.21 mM EDTA) and plated onto a 96-well plate at a density of 10⁴ cells per well in 100 μL of media. The cells were incubated at 37° C. overnight to allow them to adhere to the bottom of the well.

Experiments were run in two replicates under three different conditions. The first condition was incubation with pure DOX, the second was incubation with DOX-PCB nanoparticles with no 365 nm light exposure, and the third was incubation with DOX-PCB nanoparticles that had previously been exposed to 60 min of 365 nm light from the same light source used in the light-activated drug release characterization study.

The spiked media samples were prepared ahead of time using stock solutions of 200 μM DOX and 200 μM DOX-PCB nanoparticles in water. A sample of the pure DMEM media described above was concentrated to 80% of the original volume. The concentrated media was rediluted to its original volume using the stock solutions of drug to achieve solutions of DOX and DOX-PCB nanoparticles at 13.3 μM in the media. A ⅓ serial dilution was then performed to create concentrations that ranged from 13.3 μM to 2.0 nM. The 80% volume concentrated media rediluted to 100% volume with pure sterile water with no drug added was used as the control for each condition. The incubation media in each well was then replaced with the above prepared solutions causing minimal disturbance to the cells. The cells were allowed to incubate at 37° C. for 72 hours.

At 72 hours, the In Vitro Toxicology Assay Kit (TOX2) from Sigma Aldrich was used to perform an XTT cell viability assay. Phenol red free DMEM Media containing 10% Fetal Bovine Serum and penicillin-streptomycin from Gibco was used for the XTT assay to prevent phenol red interference with the absorption measurements. After 30 minutes of incubation time, the absorbance of each well was measured using a Tecan Infinite M200 plate reader (San Jose, Calif., USA). The collected absorbance values were used to create percent viability vs. dose curves with the PRISM 4.0 program from GraphPad Software Inc. (La Jolla, Calif., USA) using the sigmoidal dose-response (variable slope) curve fit. IC50 values were then determined from the fitted curves.

The cytotoxicity data for A549 human lung cancer cells is shown in FIG. 8. The DOX-PCB nanoparticles displayed a 30-fold reduction in cellular toxicity prior to activation as compared to pure DOX. From the cellular localization study in FIG. 7, the fluorescent DOX-PCB was found in the cytoplasm of the PTK2 cells but not in the nuclear DNA. The lack of fluorescent signal in the nucleus before 365 nm light exposure supports that the delivered DOX-PCB prodrug did not have any DNA interactions such as intercalation. This could account for the reduction in the toxicity of the DOX-PCB nanoparticles compared to free DOX. It is yet to be determined whether the endocytosed nanoparticles dissolve into free monomers within the cellular environment or whether the nanoparticles remain intact.

Upon exposure to 365 nm light pure DOX was released from the nanoparticles resulting in a reduction of the IC50. DNA intercalation of the DOX released from DOX-PCB shown in FIG. 7 would account for the sample's observed increase in cellular toxicity.

The number of DOX-PCB monomers that convert to pure DOX can be increased by using higher power LED light sources or by increasing the exposure time. The release of more DOX increases the cellular toxicity of the DOX-PCB nanoparticle sample and would result in a further reduction of the IC50.

The size and surface characteristics of the DOX-PCB nanoparticles are similar to Doxil® and it is likely that these particles would accumulate in a similar manner to Doxil® in both the tumor and in specific sites of healthy tissue including the bone marrow. However, in the case of DOX-PCB nanoparticles, pure DOX would only be released inside the 365 nm light-exposed tumor tissue. By adjusting the intensity of the 365 nm light the DOX release rate can be tailored to match that of Doxil® or could be increased to make DOX bioavailable at faster rates. The reduced toxicity of the DOX-PCB nanoparticle compared to free DOX could result in significantly reduced bone marrow toxicity as well as reduced skin ulcerations in the hands and feet which would significantly improve patient quality of life during treatment.

### Example 6

### Coating of DOX-PCB Nanoparticles

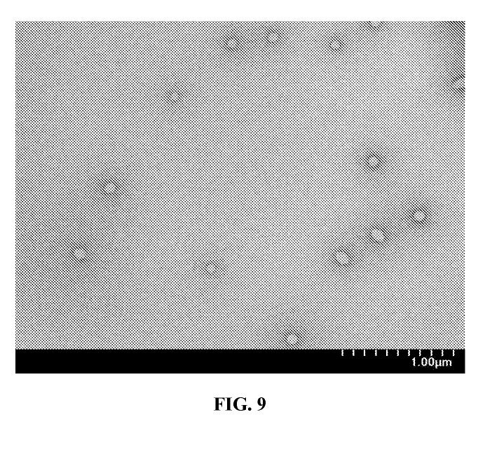

The following is an example fabrication method for a DOX-PCB prodrug nanoparticle coated with lipid and lipid-polyethylene glycol conjugate: lecithin and 1,2-diastearoyl-sn-glycero-3-phosphoethanolamine (DSPE)-PEG2000 (at a 7:3 molar ratio of lipid to lipo-polymer) at a weight ratio equal to 10% of the DOX-PCB prodrug monomer to be added are dissolved in water with 4 wt % ethanol. The lecithin/DSPE-PEG solution is heated to 65° C. and then 168 μL of the DOX-PCB prodrug dissolved in acetonitrile is added to the lecithin/DSPE-PEG solution dropwise under vortex as described earlier in the nanoprecipitation process for the DOX-PCB nanoparticles. The resulting solution is vortexed for 3 minutes followed by the addition of ultrapure water to achieve a solvent ratio of 10:1 water:acetonitrile. The sample is then stirred gently overnight while open to the atmosphere to evaporate the organic solvent. Excess lipid, lipid-polymer, or any material used in the coating process can be removed by dialysis or centrifugal filtration. Lipid coated nanoparticles made with the DOX-PCB core as described above are shown in FIG. 9.

### Example 7

### Production of PEM-PCB Prodrug

A different chemotherapeutic Pemetrexed instead of doxorubicin was used to produce a prodrug (called PEM-PCB) using a process similar to that of Example 1. However, when PEM-PCB was exposed to the activating wavelength of light the prodrug did not break apart to release the active, pure Pemetrexed, as detected by mass spec analysis. Accordingly, different photocleavable blocking groups can be carefully chosen to release pure drug and also allow the formation of self-assembled nanoparticles.

The following sections provide information on additional aspects of the disclosed technology related to ultrasound-controlled microcapillary damage. Ultrasound can be used with or without ultrasound-sensitizing particles to help specifically damage the vasculature of the tumor or other tissue of interest to enhance the extravasation of the nanoparticles into the tissue. This ultrasound can be administered before or after the injection or infusion of the nanoparticles.

Once a tumor is detected, localizing treatment to just the tumor volume can reduce systemic side effects. Localized drug deposition can be achieved by using ultrasound to damage the tumor microcapillaries. Fluid jets that are formed through the inertial cavitation of microbubbles in the ultrasound focal zone can increase the level of damage, especially if they are directed horizontally along the capillary surface. Data presented here shows that shorter distances between microbubble pairs at the time of ultrasound exposure significantly increased the chances of this horizontally-oriented jet formation.

The major challenge with chemotherapy drugs lies in the fact that most are designed to affect all rapidly dividing cells. This can be very effective for tumors that are well developed and actively growing, but for smaller tumors that are earlier in their development the cells may not be replicating nearly as fast. In these cases the drugs will have less effect on these slower growing cells requiring prolonged exposures to produce a therapeutic effect. The goal is to expose the tumor to the highest concentration of chemotherapy drugs for the longest time possible. However, the chemotherapy drugs also have systemic effects on the replication of normal non-tumor cells which creates potentially harmful short-term and long-term side effects, especially if doses are prolonged. These side effects include neutropenia which can leave cancer patients susceptible to serious infection and cause potentially detrimental delays and reductions in a patient's chemotherapy dose. Of particular concern are long-term side effects such as cardiotoxicity which can result in the appearance of cardiomyopathy and congestive heart failure after many years of latency post-treatment. As the chances of long-term cancer survival increase, these latent side effects become a major issue. Localized chemotherapeutic delivery approaches can reduce these side effects by creating elevated drug exposure in the tumor and restricting the exposure seen by healthy tissues.

One way to help deliver more therapeutic into the tumor for a longer period of time is to cause damage to the tumor capillaries. This damage and endothelial cell dislodgment can allow more drug molecules or slow-release drug delivery nanoparticles to extravasate from circulation into the tumor tissue. This extravasation enhancement is important for smaller tumors where the characteristic tumor “leaky” vasculature may not have developed, leaving these tumors with a reduced capability to accumulate drug molecules or nanoparticles via extravasation compared to larger tumors. Capillary disruption can be effectively localized by using focused ultrasound. The use of microbubbles can increase the extent of capillary damage because they cause a local dissipation of the ultrasound wave energy through the creation of an inertial cavitation shockwave. Microbubbles that undergo inertial cavitation while in contact with a rigid or flexible boundary form fluid jets that are directed perpendicularly toward the boundary surface. While the formation of this perpendicular jet can be used to create microcapillary damage, the damage could be enhanced by influencing the concentrated jet to have a directional component that is oriented horizontally along the capillary surface, affecting a larger surface area. The influence of inter-microbubble distance on this horizontal directional component of jet formation and their ability to create localized microcapillary damage are investigated. Designing microbubbles to produce this enhanced localized disruption of the capillary wall would be beneficial for many drug delivery scenarios and antitumor applications.

Microbubbles that undergo inertial cavitation while in contact with a rigid or flexible boundary form fluid jets that are directed perpendicularly toward the boundary surface. The formation of this perpendicular jet is desirable when using the microbubbles to create damage in the microcapillaries of a tumor to enhance drug extravasation and delivery. However, more damage could be caused by influencing the concentrated jet to have a directional component that is horizontal to the capillary surface allowing the jet to affect a larger surface area. This study investigated the influence of inter-microbubble distance on the horizontal directional component of jet formation through the observation of lipid debris clouds created by the destruction of the microbubble lipid monolayer. It was observed that at distances smaller than 37 μm the microbubbles began to interact with one another resulting in distorted and ellipsoid-shaped debris clouds suggesting the creation of a horizontal directional component in the jet. At inter-microbubble distances less than 10 μm, significantly elongated debris clouds were observed. These distortions show a significant distance dependent interaction between microbubbles that influences the direction of the jet. It was observed that microbubbles in physical contact with one another exclusively caused these significantly elongated debris clouds.

### INTRODUCTION

The use of microbubbles as drug delivery vehicles has become a field of considerable interest in recent years. One of the unique properties of microbubbles that distinguishes them from other drug delivery vehicles is their sensitivity to focused ultrasound. The compressibility of the gas within the microbubble allows the ultrasound to drive size oscillations creating a mechanical actuation force. At low ultrasound intensities these size oscillations result in microstreaming of fluid around the microbubble. At higher ultrasound intensities, and at the correct ultrasound frequency, resonance behavior can be achieved, resulting in size oscillations that are large enough to force the microbubble to undergo an adiabatic implosion known as inertial cavitation. This cavitation event produces a shockwave that radiates out from the microbubble itself and can affect the membranes of nearby cells. An attractive property of these shockwaves is that they can compromise the integrity of the membranes of nearby cells in a process known as sonoporation. This modification in membrane permeability can occur for periods of time that are long enough to allow nearby drugs to flow down their concentration gradient and enter the cell. Sonoporation is a widely-used in-vitro method to help facilitate gene transfection into cell populations. It is also being explored to help increase drug delivery in-vivo.

Microbubble-enhanced drug delivery can extend from the cellular level to whole capillaries. Microbubbles used as clinical ultrasound contrast agents are approximately 2.5 μm in diameter which prevents them from easily extravasating into tissues forcing them to stay mainly in the circulation. Their compressibility and small size allows them to travel through the microcapillaries. If the microbubbles are exposed to ultrasound and undergo inertial cavitation while in the capillaries they can cause capillary rupture. These ruptures can assist other drug delivery particles to extravasate from the circulation to the target tissue. This is of interest for drug delivery in cancerous tumors. Tumors naturally have a “leaky” vasculature with discontinuous endothelium that allows nanoparticle drug delivery vehicles, such as Doxil®, to passively extravasate and accumulate in tumor tissue over time. These vehicles slowly release their payloads into the tissue resulting in therapeutic effect. This extravasation effect can be enhanced by microbubble-induced capillary ruptures. This could help overcome one of the major limitations in drug delivery which is getting enough of the drug delivery vehicle into the tissue for a therapeutic effect. The ultrasound can be focused to small volumes allowing the capillary damage to be limited to the tumor tissue and leaving tissues outside the tumor unaffected.

There are two main types of microbubble inertial cavitation shockwaves that can rupture capillaries. The first is a symmetric collapse of the microbubble with a resulting radial expansion of the shockwave. The second is an asymmetric collapse of the microbubble resulting in a jet of fluid. The asymmetric collapse of microbubbles is of particular interest because it concentrates the energy into a smaller volume and can project that energy for longer distances. This can result in additional capillary damage by dislodging cells and causing endothelial cell death.

The direction of the jet can play an important role in the size of the damaged capillary surface region. When laser-created bubbles in the 2-3 mm diameter range come into direct contact with a rigid surface they undergo an asymmetric collapse pointed directly at the surface. This dissipates the energy of the jet in a radial pattern across the surface causing the bubble to appear to flatten out over the rigid surface. When these laser-created bubbles come into direct contact with a flexible surface, such as a gel, they too jet directly towards the surface. A microbubble that produces a fluid jet aimed directly at the microcapillary wall will induce limited localized damage to a single cell, or a small group of cells. Influencing the jet to have a directional component that is parallel to the capillary wall will expose a larger group of cells to the concentrated energy and cause more endothelial cell dislodgement. Finding ways to redirect the jet would be beneficial for drug delivery and antitumor applications.

Little is known about the jetting behavior of preformed microbubbles stabilized with a lipid coating that are in the clinically relevant size range of 1-3 μm in diameter. Larger microbubbles are unable to enter the microvasculature and are removed quickly from circulation. Most observations of microbubble jet formation have been conducted with uncoated laser-generated microbubbles larger than 100 μm in diameter near a rigid surface, or at the air-water interface. Asymmetric collapse with jet formation has been observed with lipid coated microbubbles in the 10-20 μm diameter range. However, microbubbles in the 1-3 μm diameter range are too small to allow for reliable direct observation of the involution of an asymmetric collapse using white light imaging. Direct observation is useful at this size scale to monitor secondary Bjerknes forces that cause translations, merging and cluster formation between microbubbles. However, the optical distortions created by the index of refraction difference between the gas and the surrounding water as well as the microbubbles' small size obscure the actual dynamics of collapse and involution that is the hallmark of jet formation. This makes directly observing the collapse dynamics and determining jetting direction in a statistically significant number of 1-3 μm microbubbles a challenge.

To overcome the limitations of direct observation at this small size scale fluorescence imaging was used to study the fluid flow that occurred as a result of the microbubble inertial cavitation event. A fluorescent dye was incorporated into the lipid monolayer that surrounded each microbubble. The inertial cavitation event fragmented the lipid monolayer into a fine debris cloud that was larger than the original microbubble and did not suffer from any optical distortions from the gas. The shape of the fluorescent debris cloud revealed details about the direction of the jet through the resulting fluid motion. The 1-3 μm diameter lipid-coated microbubbles studied here were positioned against a glass cover slip which served as a rigid surface. If these 1-3 μm diameter bubbles behaved in a similar manner to millimeter sized bubbles they too should jet towards the surface and flatten out over the glass creating a circular lipid debris cloud which could be monitored by fluorescence microscopy. The formation of a fluid jet with a strong horizontal directional component along the surface of the glass would result in an elongated debris cloud that could extend beyond the physical location of the original microbubble.

This study investigated if the direction of jetting could be influenced and directed horizontally along the surface by ensonifying microbubbles as pairs with varying inter-microbubble distances. It has been demonstrated with bubbles on the millimeter scale that bubble oscillation behavior is affected by the proximity of the bubbles to one another. Two distinct millimeter scale uncoated bubbles generated by dual laser pulses have been shown to create jets toward each other as they collapse. Microbubbles also change their oscillation behavior as they approach one another, producing chaotic type oscillations. The shape and symmetry of the fluorescent lipid debris clouds created by the two microbubbles can be used to determine if a horizontal component of the jet was produced.

### Materials and Methods

### Materials

Distearoyl phosphatidylcholine (DSPC) was purchased from Avanti Polar Lipids, Inc. (Alabaster, Ala.) and distearoyl phosphatidylethanolamine-methyl poly(ethylene glycol) MW5000 (mPEG-DSPE 5 k) was purchased from Laysan Bio, Inc. (Arab, Ala.). Dulbecco's phosphate buffered saline (DPBS) was purchased from Hyclone Laboratories Inc. (Logan, Utah). 3,3′-dioctadecyloxacarbocyanine, perchlorate (DiO) was purchased from Biotium, Inc. (Hayward, Calif.). Perfluorohexane (PFH) was purchased from Sigma-Aldrich (St. Louis, Mo.).

### Microbubble Fabrication

To fabricate lipid-coated microbubbles, stock solutions of lipid and lipophilic dye were prepared in chloroform at 20 mg/ml DSPC, 50 mg/ml mPEG-DSPE 5 k, and 1 mM DiO. 50 μl of the DSPC solution, 20 ul of the mPEG-DSPE 5 k solution, and 30 μl of the DiO were successively added to a 4 ml glass vial while vortexing. The amount of DiO in the combined solution was 2 mol % of the total lipid and dye content. The chloroform was evaporated under an argon gas stream while the solution was under vortex. This created a lipid film along the inner surface of the vial. 500 μl of DPBS was added to the film and the lipids and dye were resuspended by vortexing the vial for 15 seconds followed by heating at 75° C. for 1 minute. The cycle of vortexing and heating was repeated until the lipids were well suspended and no lipid residue was left on the vial walls. The sample was left to cool to ambient temperature.

To create the gas microbubbles, the top of the vial was first covered with parafilm to create a barrier between the gas in the vial and outside the vial. A 5 ml syringe equipped with a 22 gauge needle was used to draw up 1 ml of liquid PFH. With the plunger fully drawn back, the syringe was rotated to coat the walls with the liquid PFH and left to sit for at least 3 minutes to encourage vaporization of PFH into the air within the syringe creating an air/PFH vapor mixture. The syringe needle was then bent at a 130° angle into a hook shape and was inserted through the parafilm cover into the vial headspace. Next, the syringe was pumped 65 times into the vial headspace to inject the PFH/air mixture into the vial headspace. Care was taken to prevent any liquid PFH from entering the vial. The XL-2000 probe sonicator (QSonica LLC., Newtown, Conn.) tip was immediately inserted through the parafilm cover and positioned 1 mm below the gas/liquid interface. The probe sonicator was then operated at 25 W for 3 seconds to create microbubbles. The resulting bubbles were left to sit for at least 5 minutes before further processing.

Excess lipid and dye was removed from the microbubble sample by a washing procedure. The microbubbles were moved to a microcentrifuge tube and centrifuged at 1000 rpm for 3 minutes which caused the bubbles to float to the top of the liquid. The subnatant was partially removed and replaced with additional DPBS. This process was repeated 1-3 additional times and helped to reduce fluorescence background in the images from dye that was not associated with bubbles.

### Ultrasound Exposure

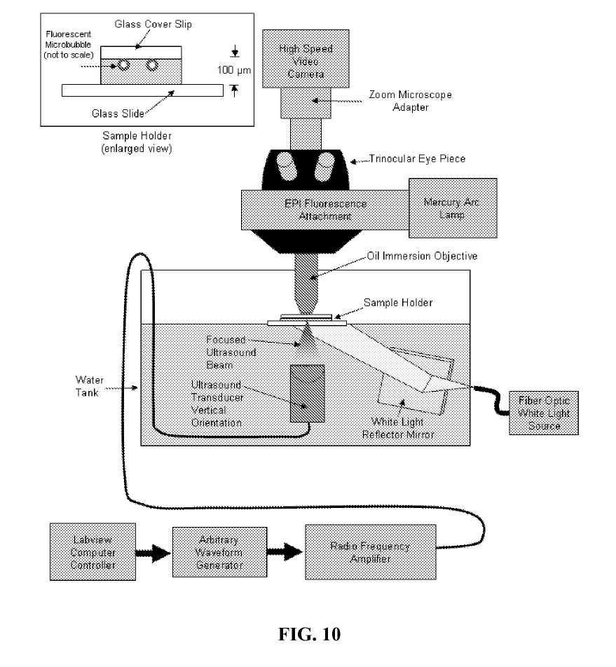

The ultrasound experiments were carried out using a custom-designed system that combined fluorescence imaging with ultrasound. A schematic of the system and the sample holder setup for these experiments is shown in FIG. 10.

A ten gallon tank of water was used to allow coupling between the ultrasound transducer and the microbubble sample. The fluorescent microbubbles in MilliQ purified water were placed in 10 μl samples on a glass microscope slide and then covered with a glass cover slip for imaging. The glass slide was then placed at the air-water interface such that just the bottom of the slide was in contact with the water. By preventing the water level from reaching the top of the glass slide, the microbubble sample was prevented from being washed into the tank water. Positioning the microbubble sample at the air-water interface allowed a 100× oil immersion objective to collect the images. The use of a high numerical aperture oil immersion objective allowed more fluorescent light to be collected from the microbubbles in order to achieve higher frame rates and allow debris cloud resolution.

The contact between the bottom side of the glass slide and the water allowed the ultrasound to travel through the water and hit the bottom of the glass slide. The ultrasound intensity was attenuated by the glass but not enough to prevent microbubble cavitation from occurring in the sample.

A 3 minute time delay between sample preparation and exposure to ultrasound allowed all the microbubbles to settle up against the glass cover slip due to their buoyancy. This ensured that there were no microbubbles in different focal planes and allowed all the microbubbles to be visible to the optical system.

The ultrasound was focused to a 1 mm² focal cross-sectional area allowing microbubbles within this region to be affected by the ultrasound pulse. The samples were scanned for microbubble pairs at different distances from one another. Once a group was identified it was centered into the ultrasound focal zone and hit with an ultrasound pulse that included a 10 ms 2.25 MHz sine wave with a pressure of 1.6 MPa (peak-to-peak). Inertial cavitation of microbubbles can begin to occur at ultrasound peak pressure amplitudes as low as 0.58 MPa. The interaction was recorded at 60 frames per second and saved for subsequent analysis.

### Video Analysis

The videos were analyzed using ImageJ software to measure the starting edge-to-edge distance of each microbubble to its nearest neighbor microbubble (“inter-bubble distance”) before ultrasound exposure. Isolated microbubbles with no other microbubbles visible in the microscope field of view were assigned the distance between the bubble edge and the edge of the field of view. The inertial cavitation event caused the fluorescent lipid monolayer on the microbubbles to fragment leaving behind a debris cloud. The dimensions of the fluorescent debris cloud for each microbubble were then measured and assigned to one of three categories. The first category was radial, where a circular pattern of debris was seen, resulting from conditions where the fluid jet was pointed normal to and directly toward the cover glass surface with little or no horizontal directional component. Debris clouds where there was less than a 1.3-fold difference between length (the longer dimension) and width were assigned to the radial category, The second category was ellipsoid where a debris cloud was produced that was between 1.3 to 2 times longer than its width, resulting from a detectable horizontal directional component in the fluid jet. The third category, denoted as “elongated” was where the inertial cavitation resulted in a large horizontal directional component of the fluid jet which distorted the debris cloud into an elongated shape such that its length was greater than 2 times the width.

### Exemplary Results

### Debris Cloud Observation

A series of still frames from the collected videos showing different microbubble debris cloud forms is shown in FIG. 11. FIGS. 11A, B and C show two microbubbles at an edge-to-edge distance of 36 μm. These microbubbles did not appear to influence one another's cavitation behavior and both created radial debris clouds. FIGS. 11D, E, and F show microbubbles at a starting distance of 8.0 μm. Some interaction appears to have occurred between the bubbles and distorted their fluorescent debris clouds into ellipsoid shapes. FIGS. 11G, H, and I show microbubbles at a starting distance of 9.3 μm. The microbubbles appear to have interacted with each other to distort both of their debris clouds into highly elongated forms that extend well beyond the original locations of the microbubbles. The debris cloud shape could not have been a result of Bjerknes forces alone since it extends beyond the original microbubble locations. The debris field could have been produced by the fluid jets having a significant directional component that was horizontal to the glass surface pushing the lipid debris out along the glass surface.

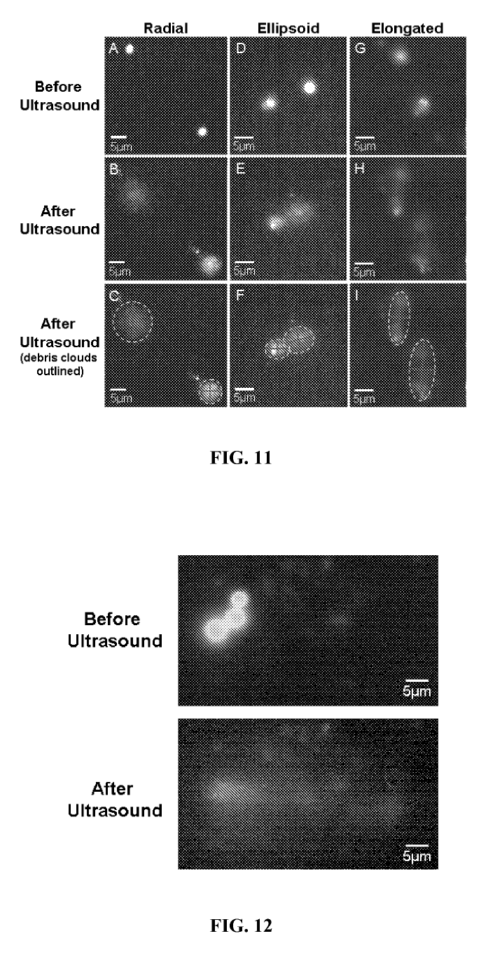

FIG. 12 shows three microbubbles that are in physical contact with one another before exposure to ultrasound. The resulting debris cloud took an elongated shape indicating a horizontal directional component of the fluid jet.

### Analysis of Debris Cloud Shape Dependence on Inter-Microbubble Distance and Microbubble Size

A total of 76 microbubbles were analyzed for debris cloud shape, microbubble size, and inter-bubble distance. The data is summarized in Table 2.

TABLE 2
Summary of microbubble data acquired from video analysis.

| | | | | Std. | Std. | 95% Confidence Interval for the Mean | | | |
|---|---|---|---|---|---|---|---|---|---|
| | | N | Mean | Deviation | Error | Lower Bound | Upper Bound | Minimum | Maximum |
| Distance | Radial | 25 | 55.5902 | 41.05703 | 8.21141 | 38.6427 | 72.5377 | 17.90 | 169.17 |
| Between | Ellipsoid | 11 | 22.1024 | 9.74174 | 2.93724 | 15.5578 | 28.6470 | 9.37 | 38.54 |
| Microbubbles | Elongated | 40 | 10.6507 | 9.66026 | 1.52742 | 7.5612 | 13.7402 | .00 | 35.90 |
| in μm | Total | 76 | 27.0909 | 31.92386 | 3.66192 | 19.7960 | 34.3858 | .00 | 169.17 |
| Microbubble | Radial | 25 | 2.6106 | .73308 | .14662 | 2.3080 | 2.9132 | 1.40 | 4.91 |
| Diameter | Ellipsoid | 11 | 2.7336 | 1.02695 | .30964 | 2.0437 | 3.4235 | 1.93 | 5.67 |
| in μm | Elongated | 40 | 4.2110 | 2.00580 | .31715 | 3.5695 | 4.8525 | 1.42 | 11.35 |
| | Total | 76 | 3.4707 | 1.73875 | .19945 | 3.0734 | 3.8680 | 1.40 | 11.35 |

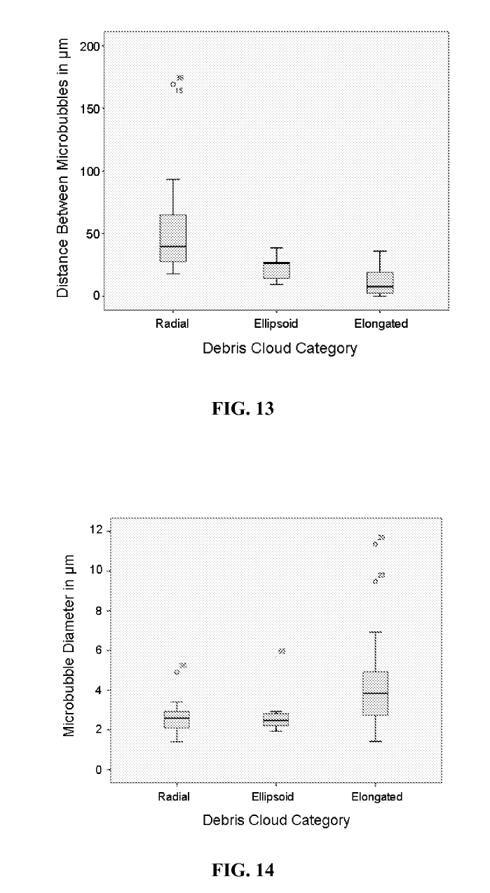

Box plots of the inter-microbubble distances for the three different cavitation debris cloud shape categories are shown in FIG. 13. The data was not normally distributed so a two-tailed nonparametric Kruskal-Wallis Test was performed at the 0.05 significance level. A significant difference was found in inter-microbubble distance between the three debris cloud shape categories (p<0.001). Post hoc comparisons using the Dunn-Sidak adjustment showed a significant difference between the inter-microbubble distances that resulted in radial debris clouds and those that resulted in elongated debris clouds (p<0.001). There was also a significant difference between the distances that resulted in ellipsoid debris clouds and elongated debris clouds (p=0.041). The difference between the microbubble distances that resulted in radial debris clouds and those that resulted in ellipsoid debris clouds trended towards significance (p=0.067). All statistical calculations were performed using IBM® SPSS® Statistics Version 21 software.

Box plots of the diameter of the microbubbles in the three different debris cloud expansion categories are shown in FIG. 14. There was no significant difference between the microbubble diameters of bubbles that produced radial and ellipsoid debris clouds (p=1) using the same statistical test described for the above comparisons of inter-bubble distance. There was a significant difference between the diameters of bubbles that produced radial debris clouds and those that resulted in elongated debris clouds (p<0.001). This difference was likely a result of the larger microbubbles being generally closer to one another than the smaller bubbles in the study population, perhaps caused by the manner in which these bubbles settled at the liquid-coverslip interface. It is possible that the larger microbubbles created flow patterns around their perimeter as they rose up in the fluid due to buoyancy forces. These flow patterns could have pulled the microbubbles together before they stopped their motion at the interface with the glass coverslip. There was also a significant difference between the diameters of bubbles that produced ellipsoid debris clouds and elongated debris clouds (p=0.009) which was likely also caused by the closer inter-bubble distance seen with larger microbubbles.

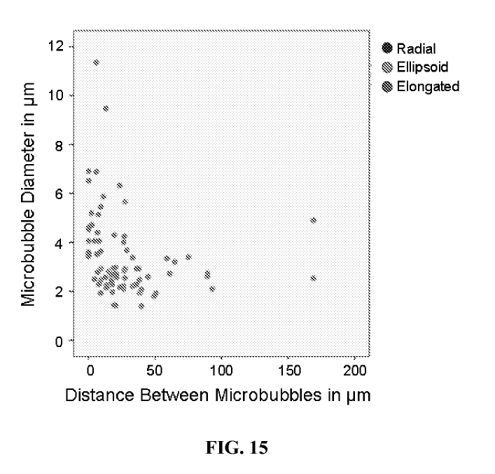

A scatter plot of the diameter of the microbubble versus the inter-microbubble distance is shown in FIG. 15 and is color coded to the three different debris cloud categories. A clear pattern can be seen where radial debris cloud expansion occurs at the larger inter-microbubble distances and ellipsoid and elongated debris clouds occur only at the smaller distances. Ellipsoid formation was observed to begin occurring at inter-microbubble distances of 37 μm. Below 10 μm in inter-microbubble distance, bubbles were seen to exclusively produce elongated debris clouds.

### Discussion of Exemplary Results

As can be seen in FIGS. 11 and 12, the distance between microbubbles had an impact on the morphology of the resulting fluorescent lipid debris cloud. These debris clouds were produced by the fragmentation of the fluorescent lipid monolayer that surrounded and stabilized the surface of the original microbubbles. The morphology of the debris cloud was highly influenced by fluid flow and revealed information about the directional components of the fluid jet resulting from microbubble collapse. In our experimental setup, the interaction between microbubbles began to distort the shape of the debris cloud at inter-microbubble distances shorter than 37 μm resulting in ellipsoid debris clouds. At an inter-microbubble distance of around 25 μm the microbubbles were interacting more extensively and the microbubble cavitation events more frequently resulted in elongated forms indicating a substantial horizontal directional component to the fluid jet. Microbubbles that had less than 10 μm of separation or were in physical contact with one another prior to ultrasound exposure were always seen to result in elongated debris clouds.

Two types of interactions were likely occurring between microbubbles in this study when ensonified with ultrasound. The first was the influence of microstreaming fluid flow around each microbubble that occurred as the microbubbles started to oscillate just before the cavitation event. The second were the secondary Bjerknes forces that caused microbubble attraction. This microbubble attraction is shown in FIGS. 11D and E. In FIG. 11D the microbubbles start at a center-to-center distance of 11.0 μm but the resulting debris clouds in FIG. 11E have a center-to-center distance of 6.5 μm indicating that the microbubbles attracted each other before they cavitated. If the microbubbles were attracted to one another just before the cavitation event then the microstreaming occurring around each microbubble could also easily influence the individual microbubble oscillations. This influence would grow in intensity as the microbubbles became closer to one another causing distortions in the collapse of the microbubble towards the glass surface and creating horizontal directional components in the jet. In this particular experimental setup, the inter-bubble distance at which the microstreaming and Bjerknes forces appeared to start to influence the microbubble oscillations and jetting direction was at 37 μm. At distances around 25 μm the microstreaming and secondary Bjerknes forces were repeatedly intense enough to create a higher incidence of horizontal directional components in the fluid jet. These distances may vary depending on microbubble lipid shell stiffness and ultrasound pressure applied.

The formation of elongated and ellipsoid debris clouds did not appear to be influenced by the size of the microbubble at sizes below 4 μm in diameter. Above 4 μm in diameter the microbubbles all had inter-microbubble distances below 30 μm except for one microbubble that was isolated and had a radial debris cloud. The population subset of microbubbles below 4 μm in diameter shows a statistically significant difference in inter-microbubble distance between those bubbles that formed elongated debris clouds and those that had radial debris clouds (p<0.001) using the same statistical test as described above for comparisons of inter-microbubble distance. However, for this same population of bubbles below 4 μm, there was no statistically significant difference in bubble size between all three groups (p=0.169). This supports that for microbubbles less than 4 μm in diameter, which is the relevant size range for clinically-used microbubbles, the more dominant factor for formation of horizontal directional components in the jet was the distance between the microbubbles and not their size.

The formation of horizontal directional components in the fluid jet can be a desirable trait because the energy is concentrated into a smaller volume and is directed at more cells, resulting in a longer distance of influence across the capillary wall. It is also more likely to cause extensive capillary ruptures and endothelial cell death which would be beneficial when trying to degrade the endothelium of tumor tissue in an effort to enhance the extravasation of subsequently administered drug or drug delivery vehicles.

Horizontal directional components in the jet could be increased significantly in the tumor region by assuring that the microbubbles are preset in sufficient concentrations so that they would be no more than 25 μm apart from one another. Achieving these high concentrations might require prohibitively high dose administration to the patient. One potential option to overcome this limitation is the incorporation of endothelial targeting agents which help the bubbles to accumulate in closer proximity Another option is tethering the microbubbles together using ligand binding which would allow them to be pre-coupled prior to administration. Using dilute concentrations of the binding agents followed by a blocking agent would allow for predominantly microbubble dimer formation and prevent microbubbles from cross-linking into larger cluster networks. This would ensure that the microbubbles were close enough to cause substantial horizontal directional components in the jet similar to the situation shown in FIG. 12.

### CONCLUSIONS

The starting distance between microbubbles prior to ultrasound exposure had a significant effect on the shape of the resulting lipid debris cloud. This debris cloud shape was influenced by directional components of the fluid jet resulting from inertial cavitation. In our experimental configuration, inter-microbubble distances greater than 37 μm resulted in jets that were directed straight at the glass interface creating circular debris clouds. This indicates that at this distance the microbubbles were not interacting with one another. At distances less than 37 μm microbubble microstreaming and secondary Bjerknes forces began to influence and distort microbubble oscillations resulting in detectable horizontal directional components creating ellipsoid-shaped debris clouds. At distances less than 10 μm significant elongation of the debris clouds was observed in every instance indicating a strong horizontal directional component was created in the fluid jet. Microbubbles that were in physical contact with one another exclusively formed these elongated debris clouds. Physically attaching microbubbles together in pairs could help ensure formation of a horizontal directional component in the jet to maximize capillary damage for in-vivo drug delivery applications.

While this patent document contains various specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document and attached appendices should not be understood as requiring such separation in all embodiments.

Only a few implementations and examples are described and other implementations, enhancements and variations can be made based on what is described and illustrated in this patent document.

1. A chemical delivery system, comprising:
a nanoparticle comprising a chemical substance that is chemically inactive and capable of being converted to a chemically active substance caused by an applied optical stimulus, wherein the nanoparticle is configured to be injected, infused, or ingested by a subject and move through the subject's bloodstream to regions of the subject's body; and
a light emitting device to emit light having a particular wavelength at a selected tissue region of the subject,
wherein exposure to the emitted light on the nanoparticle at the selected tissue region chemically activates the chemical substance to interact with the selected tissue region.

2. The system of claim 1, wherein the nanoparticle is configured to move through the subject's body via passive extravasation.

3. The system of claim 1, wherein the nanoparticle includes monomers that self-assemble into a nanoparticle form, wherein each monomer comprises hydrophobic and hydrophilic regions.

4. The system of claim 1, wherein the light emitting device includes at least one of a light emitting diode or a fiber optic detector system.

5. The system of claim 1, wherein the chemical substance includes a photo-activatable prodrug and the chemically active substance is an activated drug.

6. The system of claim 1, wherein the chemically active substance includes doxorubicin.

7. The system of claim 1, wherein the selected tissue region includes a tumor.

8. The system of claim 1, wherein the nanoparticle further comprises one or more contrast agents coupled to the surface of the nanoparticle.

9. A chemical delivery device, comprising:
a nanoparticle comprising a chemical substance that is chemically inactive and capable of being converted to a chemically active substance caused by an applied optical stimulus, wherein the nanoparticle is configured to be injected, infused, or ingested by a subject and move through the subject's bloodstream to regions of the subject's body including a selected tissue region,
wherein exposure to light having a particular wavelength on the nanoparticle at the selected tissue region of the chemically activates the chemical substance to interact with the selected tissue region.

10. The device of claim 9, wherein the nanoparticle is configured to move through the subject's body via passive extravasation.

11. The device of claim 9, wherein the nanoparticle includes monomers that self-assemble into a nanoparticle form, wherein each monomer comprises hydrophobic and hydrophilic regions.

12. The device of claim 9, wherein the chemical substance includes a photo-activatable prodrug and the chemically active substance is an activated drug.

13. The device of claim 9, wherein the chemically active substance includes doxorubicin.

14. The device of claim 9, wherein the selected tissue region includes a tumor.

15. The device of claim 9, wherein the nanoparticle further comprises one or more contrast agents coupled to the surface of the nanoparticle.

16. A delivery device, comprising:
a nanoparticle comprising monomers of an agent in an inactive form, wherein upon exposure to an optical stimulus, the agent is activated.

17. The delivery device of claim 16, wherein the nanoparticle has a diameter of less than 200 nm.

18. The delivery device of claim 16, wherein the nanoparticle has a diameter of about 100 nm.

19. The delivery device of claim 16, wherein the nanoparticle has a circulation half-life that is longer than the circulation half-life of the monomers upon administration to a subject.

20. The delivery device of claim 16, wherein the nanoparticle is entirely or substantially composed of monomers of the agent.

21. The delivery device of claim 16, wherein the nanoparticle is obtained by nanoprecipitation of the monomers of the agent.

22. The delivery device of claim 16, wherein the monomer of the agent comprises hydrophobic and hydrophilic regions.

23. The delivery device of claim 22, wherein the nanoparticle is configured to comprise a core comprising the hydrophobic regions of the monomers and a surface comprising the hydrophilic regions of the monomers.

24. The delivery device of claim 22, wherein the monomers self-assemble into the nanoparticle.

25. The delivery device of claim 16, wherein the agent in an inactive form is activated by releasing an active component upon exposure to the optical stimulus.

26. The delivery device of claim 16, wherein the agent in an inactive form includes a photoactivatable prodrug.

27. The delivery device of claim 26, wherein the prodrug comprises an active drug covalently bound to a photocleavable linker.

28. The delivery device of claim 27, wherein the prodrug comprises doxorubicin as an active component, which is covalently bound to a photocleavable linker.

29. The delivery device of claim 28, wherein the prodrug is activated upon exposure to the optical stimulus which is light having a wavelength of 365 nm.

30. The delivery device of claim 16, wherein the nanoparticle comprises a surface coating.

31. The delivery device of claim 16, wherein the optical stimulus is delivered to a subject by optic fiber or LED.

32. A method for delivering an active agent to a desired tissue, organ, or body part of a subject, comprising:
(a) systemically administering to the subject a composition comprising nanoparticles, wherein each nanoparticle comprises monomers of an agent in an inactive form, and subsequently
(b) delivering an optical stimulus to the desired tissue, organ, or body part of the subject in a controlled manner such that other tissues, organs or body parts are not exposed to the optical stimulus,
whereby the agent within the administered composition in the subject is activated upon exposure to the optical stimulus.

33. The method of claim 32, wherein the composition further comprises a pharmaceutically acceptable carrier, an excipient, a preservative, a surfactant, or a combination thereof.

34. The method of claim 32, wherein the composition is systemically administered to the subject by injection, infusion or ingestion.

35. The method of claim 32, wherein the nanoparticle has a diameter of less than about 200 nm.

36. The method of claim 32, wherein the nanoparticle has a diameter of less than about 100 nm.

37. The method of claim 32, wherein the nanoparticle has a circulation half-life that is longer than the circulation half-life of the monomers upon administration to a subject.

38. The method of claim 32, wherein the nanoparticle is entirely or substantially composed of monomers of the agent.

39. The method of claim 32, wherein the nanoparticle is obtained by nanoprecipitation of the monomers of the agent.

40. The method of claim 32, wherein the monomer of the agent comprises hydrophobic and hydrophilic regions.

41. The method of claim 40, wherein the nanoparticle is configured to comprise a core comprising the hydrophobic regions of the monomers and a surface comprising the hydrophilic regions of the monomers.

42. The method of claim 40, wherein the monomers self-assemble into the nanoparticle.

43. The method of claim 32, wherein the agent in an inactive form is activated upon exposure to the optical stimulus.

44. The method of claim 32, wherein the agent in an inactive form is a photoactivatable prodrug.

45. The method of claim 44, wherein the prodrug comprises an active drug covalently bound to a photocleavable linker.

46. The method of claim 45, wherein the prodrug comprises doxorubicin as an active component, which is covalently bound to a photocleavable linker.

47. The method of claim 46, wherein the prodrug is activated upon exposure to the optical stimulus which is light having a wavelength of 365 nm.

48. The method of claim 32, wherein the nanoparticle is coated by a surface coating.

49. The method of claim 32, wherein the optical stimulus is delivered to a subject by optic fiber or LED.
